(19) United States
(12) Patent Application Publication
(10) Pub. No.: US 2023/0220443 A1
(43) Pub. Date: Jul. 13, 2023

(54) CHARACTERIZATION OF S-ADENOSYL-L-METHIONINE-CONSUMING ENZYMES WITH 1-STEP EZ-MTASE: A UNIVERSAL COUPLED-ASSAY

(71) Applicant: ALBERT EINSTEIN COLLEGE OF MEDICINE, Bronx, NY (US)

(72) Inventors: Emmanuel Sebastien BURGOS, Bronx, NY (US); David SHECHTER, New Rochelle, NY (US)

(21) Appl. No.: 16/488,421

(22) PCT Filed: Feb. 20, 2018
(86) PCT No.: PCT/US2018/018654

§ 371 Date: Aug. 23, 2019

Related U.S. Application Data
(60) Provisional application No. 62/462,429, filed on Feb. 23, 2017.

(51) Int. Cl. C12Q 1/48; G01N 21/76; C12Q 1/34

(52) U.S. Cl. CPC C12Q 1/48; C12Y 201/01; C12Y 305/04; G01N 21/76; C12Q 1/34

(57) ABSTRACT

Methods and kits are disclosed for measuring activity of a methyltransferase or a radical SAM enzyme or for screening for an inhibitor of a methyltransferase or a radical SAM enzyme, where the methods and kits comprise, respectively, deaminase TM0936 for a MTase coupled assay and deaminase PA3170 for a radical SAM coupled assay.

### CROSS-REFERENCE TO RELATED APPLICATION

This application claims the benefit of U.S. Provisional patent application No. 62/462,429, filed on Feb. 23, 2017, the contents of which is herein incorporated by reference in its entirety.

### STATEMENT OF GOVERNMENT INTEREST

This invention was made with government support under grant number GM108646 awarded by the National Institutes of Health. The government has certain rights in the invention.

### BACKGROUND OF THE INVENTION

Throughout this application various publications are referred to in parenthesis. Full citations for these references may be found at the end of the specification immediately preceding the claims. The disclosures of these publications are hereby incorporated by reference in their entireties into the subject application to more fully describe the art to which the subject application pertains.

Protein post-translational modifications (PTM) regulate many biochemical processes (1-3). For instance, the deposition and removal of histone PTMs can govern cell fate. Methyl marks are written by methyltransferases (MTases) and fueled by a universal methyl-donor: S-adenosyl-L-methionine (SAM) (4). Small molecule methyltransferases (SMMT) were the first discovered (5,6). Further studies identified DNA methyltransferases (DNMT) as key catalysts to edit cytosine at certain CpG sequences, thus modulating cellular differentiation and transcriptional silencing (7). Later, protein lysine and arginine methyltransferases (PKMT and PRMT, respectively) emerged as crucial enzymes responsible for histone tail modifications (8,9). While methylation of lysine 4 on histone H3 (H3K4me3) is undeniably responsible for activation of transcription, establishing an universal code to translate PTMs and their cross talk is still at the early stage of development (10,11). Nevertheless, it is evident that erratic methylation patterns are implicated in oncogenesis and tumor progression (9,12,13). Overexpression of PKMTs and PRMTs in tumors is correlated with poor clinical prognosis (14-18). MTases are emerging cancer targets and they provide a novel playground for biological chemists to enhance the clinical use of personalized therapies (19-21).

Current MTase assays rely on the detection of either product of the transferase reactions, methyl marks or S-adenosyl-L-homycysteine (SAH FIG. 1). The use of radiolabeled SAM makes it possible to quantify the radioactivity incorporated within the acceptor target. Either a DNA strand or a peptide, the methylated product can be separated from methyl-donor via specifically charged filters (FIG. 1, arrows 1 and 2), solid phase extraction sorbents or liquid chromatography (22-25). Methylation can also be detected via antibody-specific recognition combined with fluorescence resonance energy transfer (FRET, AlphaLISA; FIG. 1, arrow 3) (26,27). Although low detection limits may be reached with these assays, there are major drawbacks to these methods: 1) a discontinuous approach limiting the analysis throughput, 2) the elevated costs of radioactive waste treatment along with unstable radioactive SAM and 3) the highly specific immuno-detection may limit the analysis to one single MTase.

On the over hand, SAH detection is well suited to the characterization of a wider range of MTases as SAH is the universal by-product of all transferase reactions. Therefore, multiple assays are based on this detection, either directly or through the use of recombinant coupling enzymes to catabolize SAH and channel it into a metabolite easily detectable (FIG. 1; arrows 4-12). An additional experimental benefit is that this approach relieves the MTases from product-inhibition.

Many assays have been developed to detect SAH. For instance, bacterial S-adenosyl-L-homocysteine nucleosidase (MTAN, E.C. 3.2.29) generates adenine and S-(5-deoxy-D-ribos-5-yl)-L-homocysteine (SRH). Adenine can either be detected continuously by 1) luminescence at 570 nm through efficient conversion into AMP and ATP using adenine phosphoribosyltransferase (APRT, E.C. 2.4.2.7), pyruvate phosphate dikinase (PPDK, E.C. 2.7.9.11) and firefly luciferase (FLUC, E.C. 1.13.12.7; FIG. 1, arrow 4) (28,29) or 2) decrease of absorbance at 265 nm following deamination into hypoxanthine (Hx; FIG. 1, arrow 5) using adenosine deaminase (ADA, E.C. 3.5.4.2) (30,31). Meanwhile, S-ribosylhomocysteinase (LuxS, E.C. 4.4.1.21) catabolizes SRH into L-homocysteine (Hcy-SH) for further detection of free-thiol with Ellman's reagent at 412 nm (FIG. 1, arrow 6) (32). Likewise, recombinant S-adenosyl-L-homocysteine hydrolase (SAHH, E.C. 3.3.1.1) may utilize SAH to generate Hcy-SH, later detected with thiol-sensitive reagents (e.g. ThioGlo®-1; FIG. 1, arrow 7) (33,34). In presence of ATP and adenosine kinase (AK, E.C. 2.7.1.20), the adenosine product of the SAHH reaction, is phosphorylated to AMP and further detected with a specific antibody (FIG. 1, arrow 8) (35). The remaining ATP from this kinase reaction can also be quantified by KinaseGlo® reagent (luminescence; FIG. 1, arrow 9) (36). Another approach, involving PPDK and FLUC, allows for continuous monitoring of SAH through recording of light output (FIG. 1, arrow 10) (37). A universal, yet discontinuous detection of SAH, based on competitive fluorescence polarization immunoassay (FPIA), was also achieved (38).

Finally, a very early report described a continuous assay involving a single coupling enzyme (39). The conversion of SAH into S-inosyl-L-homocysteine (SIH) was implemented to characterize the rat liver catechol O-methyltransferase through monitoring of absorbance at 263 nm (FIGS. 2A and 2B). Isolation of the deaminase from Aspergillus oryzae is likely the major drawback in using the assay (40), so the method has not been used since 1973. However, recent efforts towards the annotation of enzyme function have predicted the SAH-deaminase activity of TM0936 from Thermotoga maritina (FIG. 2A) (41). Further reports have described additional members of this enzyme family, all unable to catabolize SAM (42,43).

The present invention addresses the need for a universal and straight-forward coupled-assay for SAM-consuming enzymes (i.e., methyltransferases and radical SAM enzymes) that relies on a single enzyme.

### SUMMARY OF THE INVENTION

The invention provides methods and kits for measuring activity of a methyltransferase or a radical SAM enzyme or for screening for an inhibitor of a methyltransferase or a radical SAM enzyme, where the methods and kits comprise, respectively, deaminase TM0936 for a MTase coupled assay and deaminase PA3170 for a radical SAM coupled assay.

### BRIEF DESCRIPTION OF THE DRAWINGS

FIG. 1. The detection of methyltransfer reactions: a summary of assays currently available. The protein lysine-, arginine-, DNA and small molecule methyltransferases (PKMT, PRMT, DNMT and SMMT, respectively) deposit the methyl mark (sphere) onto specific acceptors using the universal methyl donor S-adenosyl-L-methionine (SAM). These reactions lead to the by-product S-adenosyl-L-homocysteine (SAH). In vitro, analysis of methyltransfer is achieved via two major strategies. The first approach involves methyl mark detection (methods 1-3). Then, SAH detection may be performed through both continuous and discontinuous assays (bold and dotted frames, respectively; methods 4-12).

FIG. 2A-2C. The reaction catalyzed by deaminase TM0936 and its use as a coupling enzyme for assay development. (A) The SAH by-product of methyltransferase (MTase) reactions is efficiently converted into its inosyl-derivative (SIH) by the deaminase TM0936. (B) The different spectral signatures between SAH and SIH (λmax1=259 nm and λmax2=249 nm, respectively) allow continuous monitoring of methyl transfer through UV-detection. The reactions are characterized by a decrease of absorbance at 263 nm (arrow). (C) The pH-dependence of differential extinction coefficients for the deamination reaction Δε=f(pH). UV-spectroscopy (scans 220-320 nm) was used to monitor changes in absorbance during the reaction catalyzed by TM0936. The adenosyl (A) to inosyl (I) conversion was monitored at 263 nm (black squares) while the homologous reaction using 8-aza-adenosyl (8-aza-A) was monitored at both 292 and 282 nm (black and white circles, respectively).

FIG. 3A-3C. The reaction catalyzed by deaminase PA3170 and its use as a coupling enzyme for monitoring radical SAM (RS) enzymes activity. (A) The 5′-deoxy-adenosine (5DOA) by-product of radical SAM enzyme reactions is efficiently converted into its inosyl-derivative (5DOI) by the deaminase PA3170. (B) The 5DOI to 5DOI conversion is confirmed by HPLC as these two molecules have distinct retention times. The different spectral signatures between 5DOA and 5DOI (λmax1=259 nm and λmax2=249 nm, respectively) allow continuous monitoring of methyl transfer through UV-detection. (C) The reaction catalyzed by PA3170 allows for full conversion of 5DOA into 5DOI.

FIG. 4A-4C. The drawbacks from a commercial kit. (A) The coupled-assay for MTase detection (Cayman Chemical, #700150). Through two enzymatic reactions, SAH is channeled to hypoxanthine; further oxidation by xanthine oxidase (XO) will produce uric acid and two molecules of hydrogen peroxide. The peroxide fuels horseradish peroxidase (HRP) to convert 10-acetyl-3,7-dihydroxyphenoxazine (ADHP) into fluorescent resorufin. (B) Slow channeling of SAH molecule. The coupling enzymes from the kit do not convert SAH fast enough and a 10-min lag phase is observed. (C) Channeling of SAH is not quantitative. A comparison between resorufin and SAH standard curves (squares and circles, respectively), highlighted the incomplete coupling between enzymes; nearly 50% of SAH-equivalent was lost before fluorescence emission.

FIG. 5. The linear relationship between absorbance and concentration with 1-Step EZ-MTase assay. The absorbance from different adenosine standard solutions (A; 0-1000 μM) was recorded at 263 nm with a Spectramax M5 plate reader in UV-Star 96-well flat bottom plate (Greiner Bio-One, #655801). Wells were filled with either 50-, 75-, 100- or 125-μL standard solutions (black squares, white squares, black circles and white circles, respectively). All solutions contained 10% glycerol and 1 mM μME. Absorbance/concentration relationship remains linear for absorbance below 2.

FIG. 6A-6D. Monitoring methyltransfer activities using our 1-Step EZ-MTase assay and UV-mode of detection. (A) Application to the protein arginine methyltransferase from Caenorhabditis elegans (CePRMT5). Kinetic parameters using peptide H4₍₁₋₂₀₎ were determined at pH=7.60 using 25 μM SAM and 300 nM CePRMT5: Kₘ=26±2 mM, k_{cat}=32.9±0.8 h⁻¹. (B) Application to the protein arginine methyltransferase from Trypanosoma brucei (TbPRMT7). Kinetic parameters using SAM were determined at pH=7.60 using 200 μM peptide H4₍₁₋₂₀₎ and 400 nM TbPRMT7: Kₘ=1.1±0.2 μM, k_{cat}=22.3±0.6 h⁻¹. (C) Application to the protein lysine N-methyltransferase (histone H3 lysine-9 specific) from Neurospora crassa (NcDIM-5). Kinetic parameters using peptide H3(1-s₃) were determined at pH=9.50 using 25 μM SAM and 7.6 nM NcDIM-5: Kₘ=0.9±0.1 μM, k_{cat}=30±1 min⁻¹. (D) Application to the sarcosine/dimethylglycine N-methyltransferase from Galdieria sulphuraria (GsSDMT). Kinetic parameters using SAM were determined at pH=7.80 using 100 μM MgCl₂, 5 mM sarcosine and 195 nM GsSDMT: Kₘ=95±18 μM, k_{cat}=42±2 min⁻¹. The experiments recorded at 263 nm were all performed using 4 mM final concentration of TM0936. Inserts are Z′-factors determined at multiple MTase concentrations; the lowest transferase concentration displays a Z′˜0.5, a characteristic for a good HTS assay (64).

FIG. 7A-7D. The 1-Step EZ-MTase assay is a simple tool to decipher enzymatic mechanisms. (A) The enzyme TM0936 remains a robust deaminase other a broad pH-range. Both substrates adenosine (A; black squares) and 8-aza-adenosine (8-aza-A; black circles) were assayed. Deamination rates (pmol min⁻¹) were measured under different pH conditions, at 10 μM final substrate concentration and 1 nM TM0936. (B) The pH dependence for k_{cat}/Kₘ and k_{cat} using the GsSDMT enzyme. Methyl transfer was monitored at 263 nm (pH 5.80-9.25) using sarcosine as the variable substrate (0.5-12.5 mM) and saturating levels of SAM (750 μM) with 976 nM GsSDMT and 4 mM coupling enzyme. Both log(k_{cat}/Kₘ) and log(k_{cat}) pH functions are depicted. (C) Ionic strength does not affect TM0938 activity. Deaminase activity was monitored with 10 μM adenosine at both low and high sodium chloride concentrations (0-2 M). Relative activity was arbitrary set to 100% when no salt was used. (D) High ionic strength reduces affinity between peptide substrate and the Trypanosoma brucei PRMT7 target (TbPRMT7). The Kₘ for H4₍₁₋₂₀₎ peptide was determined at saturating levels of SAM (25.8 μM) with 1 μM TbPRMT7, and 4 μM coupling enzyme at four different buffer concentrations (phosphate pH=7.60; 25, 50, 75 and 100 mM, black squares, white circles, black circles and white squares, respectively). As the buffer concentration decreases, so does the Kₘ for H4₍₁₋₂₀₎ peptide.

FIG. 8A-8D. The methyltransfer reaction catalyzed by Caenorhabditis elegans PRMT5 is sustained by the 8-aza analog of SAM. (A). A typical kinetic experiment. Transferase rates were monitored at 263 nm in phosphate buffer (pH=7.60) using various SAM concentrations (2.35-46.96 μM), saturating levels of H4₍₁₋₂₀₎ peptide (104 μM), 302 nM CePRMT5 and 4 μM coupling enzyme. Depicted rates are corrected for SAM decomposition and all adjusted to the same start absorbance. (B) Graphic representation of a kinetic experiment. Initial rates from panel A were plotted against SAM concentration and fitted to the Michaelis-Menten equation. Best fit provides both Kₘ and k_{cat} for this substrate (6.8±0.3 μM and 31.9±0.5 h⁻¹, respectively). (C) HPLC traces of a transfer reaction using 8-aza-SAM. Fueled by 8-aza-SAM (grey trace; t_{R}=4.75, λmax=280), CePRMT5 methylates H4₍₁₋₂₀₎ peptide and release 8-aza-SAH. This metabolite is instantaneously catabolized into 8-aza-SIH by TM0936 (black trace; t_{R}=11.60, λmax=254). The t_{R} retention times are in minutes, while λmax are in nm; both were determined following HPLC Method D (Cf. SEI). (D) Kinetic parameters for 8-aza-SAM against CePRMT5. Reactions were monitored at 282 nm in phosphate buffer (pH=7.60) using various 8-aza-SAM concentrations (4.16-124.8 μM), saturating levels of H4₍₁₋₂₀₎ peptide (104 μM), 1.5 mM CePRMT5 and 4 λM coupling enzyme. Initial rates were corrected for 8-aza-SAM decomposition and plotted against substrate concentration. Best fit provides Kₘ. Kₛ and k_{cat} for this substrate (35±19 μM, 67±42 μM and 15±6 h⁻¹, respectively).

FIG. 9A-9D. Sinefungin is a positive control compatible with the 1-Step EZ-MTase assay. (A) MTAN-based assays catabolize the methyltransferase inhibitor sinefungin (SIN). After one hour incubation with bacterial MTAN (1 μM, pH=6.80), the universal MTase inhibitor (120 μM; t_{R}=4.37, λmax=259) was quantitatively converted into adenine (t_{R}=12.76, λmax=260). The t_{R} retention times are in minutes, while λmax are in nm; both were determined following HPLC Method D. (B) Sinefungin is a poor substrate for TM0936. In comparison to MTAN, TM0936 (4 μM, one hour incubation) was a poor catalyst of the sinefungin deamination (t_{R}=3.08, λmax=249). (C) pH-dependence of the reaction between sinefungin and TM0936. The coupling enzyme deaminates the inhibitor very slowly under acidic conditions (pH≤6.80, 125 nM min⁻¹; pH=8.00, 760 nM min⁻¹). Reaction rates were monitored at 263 nm and carried out in 50 mM phosphate buffer with 120 μM sinefungin and 4 μM TM0936. (D) Inhibition of the sarcosine/dimethylglycine methyltransferase by sinefungin. The inhibitor (0-244 μM) along with saturating levels of SAM and sarcosine (763 μM and 5 mM, respectively) were incubated with GsSDMT (195 nM) and TM0936 (4 μM) at pH=6.80. The initial methyltransfer rates were recorded at 263 nm. Further analysis provided the inhibition constant Kᵢ (1.8±0.4 μM).

FIG. 10A-10B. The use of 8-aza-SAM and a fluorescence-mode of detection within the 1-Step EZ-MTase assay. (A) Calibration curves for the deamination of 8-aza-adenosine by TM0936. The deamination reactions using 8-aza-A (0-25 μM) were monitored through light emission at 360 nm with both 282 and 292 nm excitation wavelengths (white and black circles, respectively). Experiments were carried out at pH=5.00 (left curve fit) and pH=9.50 (right curve fit). The reaction product, 8-aza-inosine (8-aza-I), is a poor fluorophore: it does not emit light under acidic conditions and its fluorescence is 10-fold weaker than that of the 8-aza-A substrate at pH=9.50 (insert). (B) Kinetic behavior for H4₍₁₋₂₀₎ peptide against TbPRMT7 using 8-aza-SAM cofactor. The Kₘ and k_{cat} for H4 peptide were determined using the fluorescence-mode of detection.

FIG. 11A-11E. The 1-Step EZ-MTase detects glycine N-methyltransferase activity within biological samples. (A) Experimental set-up to monitor the 263 nm signal during the reaction catalyzed by human glycine N-methyltransferase (HsGNMT). Various enzyme concentrations (0-100 nM) were mixed with SAM (75 μM) and glycine (20 mM) at pH 8.00 in presence of 4 μM TM0936. (B) Compatibility between lysis buffer and the 1-Step EZ-MTase. Calibration curve were established with and without 10% v/v of buffer used for lysis of liver samples (white cross and black square, respectively). Both curves are superimposable (i.e., 619±9 nM h⁻¹ nM⁻¹ vs. 618±8 nM h⁻¹ nM⁻¹). (C) Detection of GNMT activity within rat liver extracts. Increasing volumes of extract (6-24 L) were mixed at pH 8.00 with SAM, glycine and TM0936 (75 μM, 20 mM and 4 μM, respectively). The methyltransfer (MT) catalyzed by rat GNMT is detected through loss of absorbance at 263 nm. The MT rates (μM h−1) are proportional to liver extract volumes (insert). (D) The traditional discontinuous and radioactive assay for detection of GNMT activity. Using radioactive SAM, the tritiated sarcosine product of the GNMT catalyzed reaction is isolated through solid phase extraction with charcoal. (E) GNMT activities measured within rat liver extracts. Two tissue samples from a same liver were prepared (1 and 2). GNMT activity was measured with both the radioactive (R; black bars) and the 1-Step EZ-MTase coupled assay (UV; grey bars). The effect of three freeze thaw (FTa-FTc) onto GNMT activity was evaluated.

FIG. 12A-12B. Liver extracts require saturating levels of glycine to provide optimum signal out-put during GNMT activity measurement. (A) Detection of GNMT activity with 1-Step EZ-MTase assay. Two 300 μL reactions containing liver extract (24 L), SAM (75 μM), μME (5 mM), GSH (2.5 mM), 10% glycerol and TM0936 (4 μM) were carried out at pH=8.00 (75 mM phosphate), with and without glycine (20 mM; + and − signs, respectively). Decrease of absorbance was recorded over four hours at 263 nm in a 96-well plate using 250 μL volumes. (B) Detection of GNMT activity with radioactive assay. Two 150 μL reactions containing liver extract (12 μL), SAM (75 μM; 14×106 cpm tritium), μME (5 mM), GSH (2.5 mM), 10% glycerol and TM0936 (4 μM) were carried at pH=8.00 (75 mM phosphate), with and without glycine (20 mM; + and − signs, respectively). Aliquots (15 μL) were quenched upon charcoal treatment at 10, 15, 20, 30, 45, 60, 120, 180 and 240 min; radioactive sarcosine product, was quantified by scintillation counting (cpm). Supported by both the 1-Step EZ-MTase and the radioactive assay, the endogenous glycine from liver extract is sufficient to detect GNMT activity; a 20 mM substrate concentration is required to saturate the methyltransferase. Upon glycine saturation, a 2.28- and 2.38-fold increase in GNMT activity is detected by the 1-Step EZ-MTase and the radioactive assay, respectively.

### DETAILED DESCRIPTION OF THE INVENTION

The invention provides a method of measuring the activity of a methyltransferase comprising contacting the methyltransferase with S-adenosyl-L-methionine (SAM) to generate S-adenosyl-L-homocysteine (SAH) and quantitatively catabolizing SAH to S-inosyl-L-homocysteine (SIH) in the presence of the deaminase TM0936.

The methyltransferase can be a protein methyltransferase, DNA methyltransferase or RNA methyltransferase. The methyltransferase can be, for example, a lysine methyltransferase, an arginine methyltransferase, a histone-lysine N-methyltransferase, a glycine N-methyltransferase or a sarcosine/dimethylglycine N-methyltransferase.

The reaction can be carried out at a pH between pH 5 and pH 10. The reaction can be carried out in the presence of sinefungin.

Methyltransferase activity can be quantified by measuring a decrease of absorbance at 263 nm.

The reaction can be carried out using a fluorescent SAM analog. As an example, the fluorescent SAM analog can be S-8-aza-adenosine-L-methionine (8-aza-SAM) and methyltransferase activity can be monitored through a decrease of fluorescence emission at 360 nm.

The methyltransferase activity can be measured from or within a biological sample, such as, e.g., a liver sample.

The invention also provides a method of screening for an inhibitor of a methyltransferase, comprising carrying out any of the methods of measuring the activity of a methyltransferase disclosed herein in the present and in the absence of a candidate compound, wherein a decrease in the activity of methyltransferase in the presence of the compound, compared to methyltransferase activity in the absence of the compound, indicates that the compound is an inhibitor of the methyltransferase.

The invention also provides a kit for measuring the activity of a methyltransferase, the kit comprising: S-adenosyl-L-methionine (SAM) and/or fluorescent SAM analog; and the deaminase TM0936. The fluorescent SAM analog can be, for example, S-8-aza-adenosine-L-methionine (8-aza-SAM).

The invention also provides a method of measuring the activity of a radical SAM enzyme comprising contacting the enzyme with S-adenosyl-L-methionine (SAM) to generate 5′-deoxyadenosine (5DOA) and quantitatively catabolizing 5DOA to 5′-deoxyinosine (5D01) in the presence of the deaminase PA3170.

A radical SAM enzyme is an enzyme that use a [4Fe-4S]⁺ cluster to reductively cleave S-adenosyl-L-methionine (SAM) to generate a radical, usually a 5′-deoxyadenosyl radical, as a critical intermediate.

The reaction can be carried out at a pH between pH 5 and pH 10. The reaction can be carried out in the presence of sinefungin.

The SAM enzyme activity can be quantified by measuring a decrease of absorbance at 263 nm.

The SAM enzyme activity can be measured from or within a biological sample, such as, e.g., a liver sample.

The invention also provides a method of screening for an inhibitor of a radical SAM enzyme, comprising carrying out any of the methods of measuring the activity of a radical SAM enzyme disclosed herein in the present and in the absence of a candidate compound, wherein a decrease in the activity of radical SAM enzyme in the presence of the compound, compared to radical SAM enzyme activity in the absence of the compound, indicates that the compound is an inhibitor of the radical SAM enzyme.

The invention also provides a kit for measuring the activity of a radical SAM enzyme, the kit comprising: S-adenosyl-L-methionine (SAM) and the deaminase PA3170.

In one embodiment, TM0936 (for MTase coupled assay) has the following amino acid sequence (Thermotoga maritima, SEQ ID NO:1):

MIIGNCLILKDFSSEPFWGAVEIENGTIKRVLQGE
VKVDLDLSGKLVMPALFNTHTHAPMTLLRGVAEDL
SFEEWLFSKVLPIEDRLTEKMAYYGTILAQMEMAR
HGIAGFVDMYFHEEWIAKAVRDFGMRALLTRGLVD
SNGDDGGRLEENLKLYNEWNGFEGRIFVGFGPHSP
YLCSEEYLKRVFDTAKSLNAPVTIHLYETSKEEYD
LEDILNIGLKEVKTIAAHCVHLPERYFGVLKDIPF
FVSHNPASNLKLGNGIAPVQRMIEHGMKVTLGTDG
AASNNSLNLFFEMRLASLLQKAQNPRNLDVNTCLK
MVTYDGAQAMGFKSGKIEEGWNADLVVIDLDLPEM
FPVQNIKNHLVHAFSGEVFATMVAGKWIYFDGEYP
TIDSEEVKRELARIEKELYSS.

In one embodiment, PA3170 (for radical SAM coupled assay) has the following amino acid sequence (Pseudomonas aeruginosa. SEQ ID NO:2):

MPNVRNPFDLLLLPTWIVPVEPAGVVLRDHALGIR
DGQIAVVAPREQAMRHGATEIRELPGMLLAPGLVN
AHGHSAMSLFRGLADDLPLMTWLQDHIWPAEGQWV
SEDFIRDGTELAIAEQVKGGITCFSDMYFYPQAIC
GVVHDSGVRAQVAIPVLDFPIPGARDSAEAIRQGM
ALFDDLKHHPRIRIAFGPHAPYTVSDDKLEQILVL
TEELDASIQMHVHETAFEVEQATERNGERPLARLH
RLGLLGPRFQAVHMTQVDDDDLAMLVETNSSVIHC
PESNLKLASGFCPVEKLWQAGVNVAIGTDGAASNN
DLDLLGETRTAALLAKAVYGQATALDAHRALRMAT
LNGARALGLERLIGSLEAGKAADLVAFDLSGLAQQ
PVYDPVSQLIYASGRDCVRHVWVGGRQLLDDGRLL
RHDEQRLIARAREWGPKIAASDRS.

All combinations of the various elements described herein are within the scope of the invention unless otherwise indicated herein or otherwise clearly contradicted by context.

This invention will be better understood from the Experimental Details, which follow. However, one skilled in the art will readily appreciate that the specific methods and results discussed are merely illustrative of the invention as described more fully in the claims that follow thereafter.

### EXPERIMENTAL DETAILS

### Introduction and Overview

S-adenosyl-L-methionine-consuming enzymes play a crucial role in metabolic pathways. The first subset of enzymes is composed of the methyltransferases. These proteins use SAM to deposit methyl marks. Many of these epigenetic ‘writers’ are associated with gene regulation. As cancer etiology is highly correlated with misregulated methylation patterns, methyltransferases are emerging therapeutic targets. The second subset of enzymes incorporates the radical SAM (RS) enzymes. These enzymes commonly generate a 5′-deoxyadenosyl radical; this radical is a critical intermediate and it is utilized to perform an array of unusual and chemically difficult transformations. Successful design of methyltransferase and radical SAM enzyme inhibitors relies on 1) the access to enzyme mechanistic insights and 2) the efficient screening of molecules against these targets. To characterize both methyltransferases and radical SAM enzymes (RS), we report a highly-sensitive one-step deaminase-linked continuous assay where the S-adenosyl-L-homocysteine (SAH) MTase-product or the 5′-deoxyadenosine (5DOA) radical-SAM product is rapidly and quantitatively catabolized to S-inosyl-L-homocysteine (SIH) or 5′-deoxyinosine (5DOI), respectively.

A coupled assay relying on one single enzyme is a clear asset, thus we took advantage of enzyme TM0936. This SAH-deaminase is a robust and efficient catalyst (41,43). By converting SAH into SIH, the enzyme relives MTAses from product inhibition (39,44). We coupled this catalyst to several MTase families: two protein arginine methyltransferases (PRMT5 and PRMT7), a histone-lysine N-methyltransferase (DIM-5) and a sarcosine/dimethylglycine N-methyltransferase (SDMT). We present efficient measurement of kinetic parameters well suited to high-throughput screening (HTS). Furthermore, we measured the inhibition value of sinefungin against SDMT, thus establishing the compatibility between sinefungin and TM0936 and demonstrating its use for inhibitor screening.

TM0936 is a strong catalyst and pH variations only affected the deaminase activity to a small extent; the decrease of absorbance at 263 nm was monitored accurately across a 5-unit pH range (5.0-10.0). Thus, in a technical tour de force, we established the pH dependence of SDMT reaction rates. Likewise, variations in salt concentration had no effect on TM0936 activity, and we quantified the impact of ionic strength onto the affinity between histone tails and their MTase target. Finally, conscious that a UV-mode of detection may limit the applications of this assay, we synthesized a fluorescent SAM analog. In most cases, the S-8-aza-adenosine-L-methionine (8-aza-SAM) was a good substrate for MTases. As TM0936 efficiently converted 8-aza-SAH into the non-fluorescent product 8-aza-SIH, PRMT7 activity was monitored through a decrease of fluorescence emission at 360 nm.

Unlike discontinuous radioactive- and antibody-based assays, our assay provides a simple, versatile and affordable approach towards the characterization of methyltransferases. With Z′-factors above 0.75, detectable methylation rates as low as 2 μM h⁻¹ and the use of submicromolar methyltransferase concentrations, this assay is well suited to high-throughput screening and may promote the identification of novel therapeutics.

### Materials and Methods

### Reagents

Common chemicals and reagents were obtained from commercial sources and used without further purification. D-luciferin (Gold Biolotechnology, #L-123), phosphoenolpyruvic acid (MP Biomedicals, #02151872), S-adenosyl-L-methionine (Sigma-Aldrich, #A7007; purification onto weak cation exchanger column—HiTrap CM Sepharose FF—is required to eliminate both S-adenosyl-L-homocysteine and 5′-deoxy-5′-methylthioadenosine impurities) (68), 8-aza-adenosine (Berry & Associates, #PRA10007), sinefungin and sarcosine (Santa Cruz Biotechnology; #sc-203263 and #sc-204262, respectively).

### Analytical Procedures.

HPLC methods-All purifications used a Luna C18(2) reversed phase column (Phenomenex #00G-4252-E0; 4.6×250 mm, 5 μm, 100 A), with:

Buffer B1: 100 mM potassium dihydrogenophosphate with 8 mM tetrabutylammonium bisulfate in water (pH=6.00; KOH);
Buffer B2: 100 mM potassium dihydrogenophosphate with 8 mM tetrabutylammonium bisulfate in 30% acetonitrile (pH=6.00; KOH); Buffer B3: water;
Buffer B4: 30% acetonitrile;
Buffer B5: 0.1% formic acid;
Buffer B6: 0.1% formic acid in 50% acetonitrile;
Buffer B7: 100 mM acetic acid with 100 mM triethylamine (pH=6.00; formic acid);
Buffer B8: 100 mM acetic acid with 100 mM triethylamine in 30% acetonitrile (pH=6.00; formic acid)

| time | flow | Method A | | Method B | | time | flow | Method C | | time | flow | Method D | |
|---|---|---|---|---|---|---|---|---|---|---|---|---|---|
| ( [symbol] ) | ( [symbol] ) | B1 | B2 | B3 | B4 | ( [symbol] ) | ( [symbol] ) | B5 | B6 | ( [symbol] ) | ( [symbol] ) | B7 | B8 |
| 0 | 1 | 98 | 2 | 98 | 2 | 0 | 1 | 100 | 0 | 0 | 1 | 98 | 2 |
| 5 | 1 | 98 | 2 | 98 | 2 | 8 | 1 | 100 | 0 | 6 | 1 | 98 | 2 |
| 25 | 1 | 0 | 100 | 0 | 100 | 14 | 2 | 10 | 90 | 16 | 1 | 0 | 100 |
| 26 | 2 | 0 | 100 | 0 | 100 | 14.5 | 2 | 10 | 90 | 16.5 | 2 | 0 | 100 |
| 32 | 2 | 0 | 100 | 0 | 100 | 20.5 | 2 | 10 | 90 | 19.5 | 2 | 0 | 100 |
| 33 | 2 | 98 | 2 | 98 | 2 | 21 | 2 | 100 | 0 | 20 | 2 | 98 | 2 |
| 39 | 2 | 98 | 2 | 98 | 2 | 29 | 2 | 100 | 0 | 23 | 2 | 98 | 2 |
| 40 | 0 | 98 | 2 | 98 | 2 | 30 | 0 | 100 | 0 | 24 | 0 | 98 | 0. |

[symbol]

indicates data missing or illegible when filed

NMR spectroscopy—¹H (¹¹C) NMR spectra were recorded on a Bruker Avance IIIHD 600 MHz (150 MHz) system equipped with a 5 mm H/F-TCI CryoProbe. ICON-NMR software (Bruker Biospin) was used to record all spectra. The ¹H-¹H spectra were obtained at a constant temperature of 298 K using a COSY pulse sequence: 2048 and 128 points, sweep widths of 12 and offset 5.0 ppm in the primary and secondary direction, respectively and 4 scans. The ¹H-¹³C edited spectra were obtained at a constant temperature of 298 K using the HSQC pulse sequence: with 2048 and 256 points, sweep widths of 12 and 200 ppm and offset 5.0 and 75 ppm for the ¹H and ¹³C dimension, respectively and 16 scans. The length of standard proton pulse to achieve a 90° nutation was determined with the ‘pulsecal’ command. The data were processed with Topspin 3.2 (Bruker Biospin) with baseline and phase correction. ³¹P NMR spectra were recorded on a Bruker Avance IIIHD 300 MHz (121 MHz) system equipped with a 5 mm BBFO probe.

Mass spectrometry—Accurate mass measurements were performed using the Orbitrap Velos Mass Spectrometer (ThermoFisher) in the positive ionization mode at a resolution of 60,000 (at m/z 300) and using angiotensin as the lock mass. Flow injection analysis of 50% acetonitrile/water containing 0.1% formic acid was used to introduce the sample into the mass spectrometer at 0.075 mL min⁻¹. Prior to injection of 10 μL, 10 μL of a 10 μM solution of angiotensin in 50% acetonitrile/water containing 0.1% formic acid was mixed with 20 μL of a 50 μM solution of sample in water.

### Enzymatic Syntheses

One-pot preparation of 8-aza-adenosine triphosphate (8-aza-ATP)—A 1.5-mL reaction mixture (100 mM TRIS/HEPES, 50 mM KCl, 40 mM MgCl₂, 2 mM μME, 450 mM phosphoenolpyruvate and 400 U pyruvate kinase/myokinase; pH=7.50) was carried out in a 2-mL tube containing 50 mg 8-aza-adenosine suspension (8-aza-A, C≈125 mM) and 1 mM ATP. Reaction started upon addition of adenosine kinase from Anopheles gambiae (AgAK; 25 μM final concentration, enzyme:nucleoside is 1:5,000). Samples were mixed with a LabRoller™ rotator; within an hour, precipitate (8-aza-A) dissolved as monophosphorylation occurred. After 7 h (25° C.), reactions were stopped (Amicon Ultra-0.5 mL, MWCO 10K, 10,000 rpm; 4° C., 15 min) and 8-aza-ATP (95% conversion) was isolated by HPLC (Method A-tR(min): 8-aza-ATP 19.22, 8-aza-adenosine 12.77). The fraction containing 8-aza-ATP was freeze-dried and further desalting was performed (HPLC; Method B). A last lyophilization step offered pure 8-aza-ATP. ¹H NMR δ (600 MHz, D₂O) 4.17 (2H, m, 5′-H), 4.43 (1H, q, 4′-H, J 4.2), 4.76 (1H, t, 3′-H, J 4.8), 5.16 (1H, t, 2′-H, J 4.8), 6.34 (1H, d, 1′-H, J 4.2), 8.36 (1H, s, 2-H); ³¹P NMR δ (121 MHz, D20) −23.13 (t, P0, J 19.7), −11.40 (d, Pa, J 19.6), −9.67 (br s, Pγ); ¹H-¹³C edited HSQC NMR δ (150 MHz, D₂O) 65.2 (C-5′), 70.4 (C-3′), 73.2 (C-2′), 84.0 (C-4′), 88.2 (C-1′), 157.2 (C-2). FTMS+p ESI m/z 508.9981 (M+), calcd for [C₉H₁₆N₆O₁₃P3]+508.9983.

Preparation of S-8-aza-adenosyl-L-methionine (8-aza-SAM)-A 1-mL reaction mixture (50 mM HEPES, 50 mM KCl, 10 mM MgCl₂, 1 mM μME, 25 mM phosphoenolpyruvate and 5 U pyruvate kinase/myokinase; pH=7.50) was carried out with 25 mM L-methionine and 10 mM of 8-aza-ATP. The synthesis started upon addition of methionine adenosyltransferase from Methanococcus jannaschii (MjMAT; 25 μM final concentration, enzyme: triphosphate 1:400). After 2 h at 35° C., reactions were stopped (Amicon Ultra-0.5 mL, MWCO 10K, 10,000 rpm; 4° C., 15 min) and the SAM analog (65% conversion) was isolated by HPLC (Method C-tR(min): 8-aza-SAM 4.67). The SAM analog-containing fraction was freeze-dried and further purified onto a weak cation exchanger column (HiTrap CM Sepharose FF, GE Healthcare Life Sciences, #17-5155-01; column was pre-charged with Na+, impurities—including excess L-methionine—were eluted with water while 8-aza-SAM was stripped-off column with a 200 mM HCl solution). A final lyophilization step offered pure 8-aza-SAM as white powder. ¹H NMR δ (600 MHz, D₂O; diluted sample) 2.33 (2 H, m, β-H), 2.90 (3H, s, S(R)Me; 5%), 2.94 (3H, s, S(S)Me; 95%), 3.55 (2H, m, 5′-H), 3.78 (1H, m, α-H), 3.90 (2H, m, γ-H), 4.66 (1H, dd, 4′-H, Ja 6.6, Jb 5.4), 4.79 (1H, m, 3′-H), 4.95 (1H, dd, 2′-H, Ja 2.4, Jb 1.8), 6.47 (1H, d, 1′-H, J 1.8), 8.39 (1H, s, 2-H); ¹H-¹³C edited HSQC NMR δ (150 MHz, D20; concentrated acidic sample) 23.6 (SMe), 24.7 (Cβ), 38.6 (C-5′), 44.6 (Cγ), 51.8 (Cα), 73.3 (C-3′), 73.9 (C-2′), 78.9 (C-4′), 90.3 (C-1′). FTMS+p ESI m/z 400.1398 (M⁺), calcd for [C₁₄H₂₂N₇₀O₅S]+400.1398.

Preparation of S-8-aza-adenosyl-L-homocysteine (8-aza-S4H)—The 7-mL reaction mixtures. (100 mM TRIS, 100 μM MgCl₂, 1 mM βME, 10% glycerol; pH=7.90) were carried out with 40 mM sarcosine and 0.5 mM of 8-aza-SAM. The methyltransfer reactions started upon addition of GsSDMT enzyme (10 μM final concentration; high excess to prevent product inhibition). After 1 h at 25° C., reactions were stopped (Amicon Ultra-15 mL, MWCO 10K, 4,000 rpm; 4° C., 20 min) and 8-aza-SAH (95% conversion) was isolated by HPLC (Method D-tR(min): 8-aza-SAM 4.75, 8-aza-SAH 13.07). The 8-aza-SAH fractions were freeze-dried to offer white powder. ¹H NMR δ (600 MHz, D₂O) 2.02 (2H, m, β-H), 2.60 (2H, t, γ-H, J 7.8), 2.95 (2H, dd, 5′-H, Ja 14.4, Jb 4.8), 3.74 (1H, m, α-H), 4.39 (1H, dd, 4′-H, Ja 7.2, Jb 4.8), 4.66 (1H, t, 3′-H, J 4.8), 5.13 (1H, dd, 2′-H, Ja 4.8, Jb 3.6), 6.35 (1H, d, 1′-H, J 3.6), 8.35 (1H, s, 2-H); ¹H-¹³C edited HSQC NMR δ (150 MHz, D20) 27.6 (Cγ), 30.3 (Cβ), 33.4 (C-5′), 53.7 (Cα), 73.1 (C-3′), 73.6 (C-2′), 84.0 (C-4′), 88.7 (C-1′), 156.9 (C-2). FTMS+p ESI m/z 386.1233 (M+), calcd for [C₁₃H₂₀N₇O₅S]+386.1241 and 408.1058 (M−H+Na+), calcd for [C₁₃H₁₉N₇NaO₅S]+408.1061.

Preparation of S-8-aza-inosyl-L-homocysteine (8-aza-SIH)—The 1-mL reaction mixture (100 mM HEPES, 1 mM βME, 10% glycerol; pH=7.50) was carried out with 5 mM of 8-aza-SAH. The deaminase reaction started upon addition of TM0936 (50 μM final concentration). After 15 min at 35° C., reaction was stopped (Amicon Ultra-0.5 mL, MWCO 10K, 10,000 rpm; 4° C., 15 min) and 8-aza-SIH (100% conversion) was isolated by HPLC (Method D-tR(min): 8-aza-SIH 11.60, 8-aza-SAH 13.07). The 8-aza-SIH fraction was freeze-dried to offer white powder. ¹H NMR δ (600 MHz, D20) 2.04 (2H, m, —H), 2.61 (2H, t, γ-H, J 7.8), 2.96 (2H, dd, 5′-H, Ja 14.4, Jb 4.8), 3.74 (1H, m, α-H), 4.39 (0H, dd, 4′-H, Ja 5.4, Jb 4.8), 4.65 (1H, t, 3′-H, J 5.4), 5.10 (1H, t, 2′-H, J 3.6), 6.37 (1H, d, 1′-H, J 3.6), 8.33 (1H, s, 2-H); ¹H-¹³C edited HSQC NMR δ (150 MHz, D20) 27.5 (Cγ), 30.2 (Cβ), 33.4 (C-5′), 53.8 (Cα), 73.1 (C-3′), 73.7 (C-2′), 84.2 (C-4′), 89.1 (C-1′). FTMS+p ESI m/z 387.1081 (M+), calcd for [C₁₃H₁₉N₆O₆S]+387.1081 and 409.0897 (MH+Na+), calcd for [C₁₃H₁₈N₆NaO₆S]⁺ 409.0901.

### Purification of Enzymes

Photinus pyralis luciferase (FLUC)—The original wild-type firefly luciferase gene (pQE30-FLUC) was sub-cloned into the pMCSG10 vector to offer N-terminal His₆-GST-TEVtagged enzyme (69). BL21(DE3) cells, transformed with this new vector, were grown at 37° C. in LB broth containing 100 μg mL⁻¹ ampicillin to an OD₆₀₀=0.6. The cultures were cooled down (18° C.), supplemented with IPTG (0.4 mM final concentration) and incubated for an additional 12 h. Harvested cells were re-suspended (50 mM TRIS, 300 mM NaCl, 5 mM imidazole, 1 mM βME, 10% glycerol, pH=8.00 containing PMSF and RNAse). Lysis was achieved using BugBuster® 10× and sonication; debris were removed by centrifugation at 20,000 rpm for 20 min. The supernatant was loaded onto Ni-NTA agarose and enzyme was eluted using an imidazole gradient (5-500 mM). Fractions containing the tagged FLUC were concentrated, desalted, and further digested by His6-tagged TEV protease (4° C., 16 h). Subtractive Ni-NTA and further purification on Superdex 200 (16/300) column (50 mM TRIS, 300 mM NaCl, 1 mM βME, 10% glycerol, pH=8.00) offered FLUC. The concentrated enzyme (150 μM) was kept at −80° C.

Proteins for enzymatic syntheses—For the synthesis of 8-aza-ATP and 8-aza-SAM, the N-terminal His6-tagged adenosine kinase from Anopheles gambiae (AgAK) and methionine adenosyltransferase from Methanococus jannaschii (MjMAT) were expressed and purified as reported previously (70-72). The sarcosine/dimethylglycine N-methyltransferase from Galdieria sulphuraria (GsSDMT; DNASU clone ID GsCD00383580) was used for the preparation of 8-aza-SAH, by-products of this methyltransferase reaction with sarcosine and corresponding 8-aza-SAM (73).

Methyltransferases—The PRMT5 from Caenorhabditis elegans (CePRMT5) was expressed and purified as described previously with some modification (74,75). In addition to Triton X-100 (1%), prior use of BugBuster® reagent for cell lysis also improved yield of recovered protein. The PRMT7 from Trypanosoma brucei (TbPRMT7) was prepared using a published protocol (76). The H4₍₁₋₂₀₎ peptide substrate for CePRMT and TbPRMT7 was purchased from GenScript (>95%). Following the described procedures, the histone H3 Lysine-K9 methyltransferase from Neurospora crassa (NcDIM-5) was expressed and purified as a GST-tagged enzyme (77). The H3₍₁₋₅₃₎ peptide substrate for DIM-5 was expressed as a His₆-tagged peptide from a modification of the original vector with a stop codon (Y54Stop) (78). Ni-NTA purification, followed by removal of N-terminus tag with His₆-TEV protease (4° C., 16 h) and subtractive Ni-NTA offered H3₍₁₋₅₃₎. Peptide was purified by HPLC (78), and further lyophilization offered suitable substrate for DIM-5 kinetic studies. The sarcosine/dimethylglycine N-methyltransferase from Galdieria sulphuraria (GsSDMT; DNASU clone ID GsCD00383580) was expressed as a His₈-MBPtagged enzyme (maltose binding protein). Further removal of the His₈-MBP-tag with His6-tagged TEC protease (4° C., 16 h) and gel filtration step offered purified enzyme (73).

TM0936 coupling enzyme—The SAH-deaminase from Thermotoga maritima (gene TM0936; DNASU clone ID TmCD00084735) was expressed and purified as described in a previous report (79). Likewise, the 5DOA-deaminase from Pseudomonas aeruginosa (PA3170) was expressed and purified as described in a previous report (42). Finally, the preparation of 5′-methylthioadenosine/S-adenosyl-L-homocysteine nucleotidase from Salmonella enterica (SeMTAN) was previously reported (80).

pH-, Salt- and DMSO-Dependence of FLUC Luminescence

Relative enzymatic efficiency of FLUC upon pH variation—The impact of pH onto enzymatic efficiency for recombinant FLUC was determined at 25° C. by monitoring luminescence (RLU, relative light unit) with a SpectraMax L instrument (one photomultiplier, ‘photo-counting’ mode; Molecular Devices). Briefly, 90-μL reactions containing buffer (50 mM phosphate; pH 5.80-8.00), 1 mM MgCl₂, 1 mM βME, 10% glycerol, 10 μM of both ATP and luciferin, were loaded onto a 96-well half-area flat bottom plate (Corning, #3992). Light production started upon addition of FLUC recombinant enzyme (10 μL at 10 nM) and RLU were recorded. For optimum experimental condition (i.e. pH=8.00), the RLU was set to 1.0 and corresponding ratios were determined for other experiments.

Salt and DMSO effect on FLUC luminescence—Using the same experimental procedure as above (50 mM phosphate, pH=8.00), luminescence was recorded under various concentrations of either NaCl (0-500 mM) or DMSO (0-7.5%, v/v). Light production started upon addition of FLUC recombinant enzyme (10 μL at 10 nM) and RLU were monitored. For optimum experimental condition (i.e. no salt, no DMSO), the RLU was set to 1.0 and corresponding ratios were determined for other experiments.

Spectral analysis of the FLUC luminescence—The spectral components of FLUC luminescence were observed at various pHs (50 mM phosphate buffer) by mixing ATP (1 mM), luciferin (1 mM), MgCl₂ (5 mM), 1 mM βME, 10% glycerol and FLUC (1 μM) in a 96-well half-area flat bottom plate. Lower pH values drastically affect the enzyme and shift its luminescence towards a red emission spectrum, with light production no longer occurring below pH=6.00. A semi-quantitative analysis of the FLUC luminescence (pHs 8.00, 7.20 and 6.40) was performed under the same conditions using a quartz cuvette and a FluoroMax-3 fluorometer (3-mL reaction volume; emission scan: 450-750 nm, 5 nm s⁻¹; excitation slit width ‘shutoff’, emission slit width 10 mu). The light generated upon luciferin oxidation was a combination of three spectral components with maximum emission at 556, 606 and 654 nm; thus, experimental traces RLU=f(λ) were fitted to a three-component Gaussian function (Eq. 1) (81):

$$RLU = {RLU}_{556}^{MAX} {e}^{- 0.5 {( \frac{λ - 556}{{σ}_{556}} )}^{2}} + {RLU}_{606}^{MAX} {e}^{- 0.5 {( \frac{λ - 606}{{σ}_{606}} )}^{2}} + {RLU}_{654}^{MAX} {e}^{- 0.5 {( \frac{λ - 654}{{σ}_{654}} )}^{2}}$$

where λ is the scanned emission wavelength (FLUC), RLU^{MAX} and σ are the luminescence intensity and the Gaussian RMS width at the specific maximum emission wavelength (i.e. 556, 606 or 654 mu), respectively.

### Differential Extinction Coefficients (Δε) for Deaminase Reaction

Nucleoside concentrations (stock solutions) were determined using known extinction coefficients for: adenosine, 259(ε/M⁻¹ cm⁻¹ 15,400) and 8-aza-adenosine, 277(e/M-1 cm⁻¹ 17,830). The deaminase reactions using these various substrates were performed in quartz cuvettes (1-cm light path; 3-mL reaction volume) and absorbance measurements were carried out with a NanoDrop 2000c (scan mode: 220-320 nm with multi scans superimposed; 37° C. and continuous stirring). Briefly, the 3-mL reactions contained 50 mM buffer (acetate: pH 4.00-5.00; MES: pH 5.00-6.50; phosphate: pH 5.80-8.00; TRIS: pH 7.50-8.50; CHES: pH 8.90-9.90), 1 mM βME and 10% glycerol. Five different concentrations of either adenosine or 8-aza-adenosine substrates were used (10, 20, 30, 40 and 50 μM). Mixtures were blanked before acquisition and reactions started upon addition of deaminase (TM0936, 2 μL of 240.5 μM enzyme stock). Over the course of the deaminase reaction (e.g. adenosine→inosine), optimum wavelengths at which the highest absorbance shift is observed were selected (i.e. adenosine reaction, λ=263 mu; 8-aza-adenosine reaction, λ1=282 nm and λ2=292 nm). Scans were recorded until the absorbance at these monitored wavelengths reached a stable value (82). For each pH condition, the end-point absorbance shifts were plotted against initial nucleoside concentrations and fitted to a linear regression: each slope is a differential extinction coefficient (Δε) for deamination of the specific nucleoside substrate. Furthermore, the pH-dependence of differential extinction coefficient (Ae) for this deaminase reaction was obtained by plotting the pairs (Δε; pH) and fitting these to the following supplementary Eq. 2:

$$Δε = {Δε}_{HIGH pH} + \frac{{Δε}_{LOW pH} - {Δε}_{HIGH pH}}{1 + {10}^{( pH - {pK}_{a} )}}$$

where Δε_{HIGH pH} and Δε_{LOW pH} are the extinction coefficient measured at optimum wavelengths (i.e. λ for adenosine and λ1 or λ2 for 8-aza-adenosine) for the deaminase reaction at high and low pH, respectively, and −pKa is the logarithm of acid dissociation constant for either inosine or 8-aza-inosine. In summary: adenosine→inosine, 263(Δε_{HIGH pH}; Δε_{LOW pH}/M⁻¹ cm⁻¹ −4,655±186; −8,076±80), pKa=8.72±0.09; 8-aza-adenosine→8-aza-inosine, 282(Δε_{HIGH pH}; Δε_{LOW pH}/M⁻¹ cm⁻¹ −2,026±174; −14,975±129), pKa=7.29±0.03, 292(Δε_{HIGH pH}; Δε_{LOW pH}/M⁻¹ cm⁻¹ −3,016±119; −10,117±87), pKa=7.30±0.03.

### Z′-Factor

The screening window coefficient (Z′-factor) was determined as previously described using various CePRMT5, TbPRMT7, NcDIM-5 and GsSDMT enzyme concentrations (116-1000 nM, 139-1003 nM, 4.28-23.8 nM and 19.5-244 nM, respectively) with saturating concentrations of SAM (25.0, 25.1, 25.8 and 763 μM, respectively) and saturated levels of matching acceptor (acceptor/μM: H4₍₁₋₂₀₎>/200, H4₍₁₋₂₀₎/200, H3₍₁₋₅₃₎/315.8 and sarcosine/5,000, respectively) at either pH=7.60 (50 mM phosphate), pH=7.60 (50 mM phosphate), pH=9.50 (50 mM glycine) or pH=7.80 (50 mM phosphate), respectively (83). Each experiment consisted of 8 sample replicates (s; with both SAM and acceptor included in reactions) and 8 control replicates (c; with SAM and without acceptor in reactions). Statistical analysis of the initial rates using the following supplementary Eq. 3 yields the corresponding Z′-factor:

$${Z}^{′} = 1 - \frac{3 {σ}_{s} + 3 {σ}_{c}}{\vert {μ}_{s} - {μ}_{c} \vert}$$

where μs is the mean value of the initial rates for samples s and as is the standard deviation of the initial rates for samples s (3σₛ corresponds to a 99.73% confidence interval).

### UV-Based Coupled Assay for MTases

Kinetic behavior for peptide H4₍₁₋₂₀₎ against CePRMT5 (SAM sat.)—Briefly, kinetic parameters were determined at 20° C. in a UV-Star 96-well flat bottom plate (Greiner Bio-One, #655801) by continuous monitoring of absorbance at 263 nm using a SpectraMax M5 instrument (Molecular Devices). The 250-μL reaction mixtures from a same row contained 50 mM phosphate buffer pH=7.60, 1 mM βME, 10% glycerol, 4 μM coupling deaminase TM0936, 300 nM CePRMT5 and various peptide concentrations (5-180 μM; in the first well, peptide acceptor was omitted to account for background signal—natural decomposition of SAM). The methyltransfer reactions started upon addition of SAM (25 μM saturating final concentration) and absorbance was recorded. Initial rates were first corrected for background signal, then plotted against H4₍₁₋₂₀₎ concentrations and fitted to the Morrison kinetic model (Eq. 4) to yield corresponding kinetic parameters (Kₘ, k_{cat}) (84):

ν=0.5 k_{cat}(([MTase]+[H4]+Kₘ)−\sqrt{([MTase]+[H4]+Kₘ)²−4[MTase][H4]})

where [MTase] and [H4] are the total concentration of methyltransferase and peptide acceptor, respectively; Kₘ is the Michaelis constant and k_{cat} is the turnover for H4₍₁₋₂₀₎ substrate.

Kinetic behavior for SAM against CePRMT5 (H4₍₁₋₂₀₎ sat.)—Briefly, the measurements consisted of two experimental subsets: MTase reactions (both SAM and peptide added) and MTase blanks (SAM was added without peptide to account for natural SAM decomposition). The 250-μL reaction mixtures from a same row contained 50 mM phosphate buffer pH=7.60, 1 mM βME, 10% glycerol, 4 μM coupling deaminase TM0936, 300 nM CePRMT5 and various SAM concentrations (2.35-47.0 μM). Absorbance at 263 nm was recorded after addition of either H4₍₁₋₂₀₎ (104 μM saturating final concentration; MTase reactions) or water (MTase blanks). Initial rates were first corrected for background signal, then plotted against SAM concentrations and fitted to the Morrison kinetic model to yield corresponding kinetic parameters.

Kinetic behavior for peptide H4₍₁₋₂₀₎ against TbPRMT7 (SAM sat.)—Experiments were conducted following matching CePRMT5 protocol above. Briefly, the 250-μL reaction mixtures from a same row contained 25 mM phosphate buffer pH=7.60, 1 mM βME, 10% glycerol, 4 μM coupling deaminase TM0936, 1.0 μM TbPRMT7 and various peptide concentrations (16.6-243.3 μM; in the first well, peptide acceptor was omitted to account for background signal—natural decomposition of SAM). Reactions started upon addition of SAM (25.8 μM saturating final concentration) and absorbance was recorded. Initial rates were first corrected for background signal, then plotted against H4₍₁₋₂₀₎ concentrations. Fit to the Michaelis-Menten model (Eq. 5) provided corresponding kinetic parameters:

$$v = {k}_{cat} \frac{[ MTase ] [ H 4 ]}{{K}_{m} + [ H 4 ]}$$

where [MTase] and [H4] are the total concentration of methyltransferase and H4 peptide, respectively; Kₘ is the Michaelis constant and k_{cat} is the turnover for H4 substrate.

Kinetic behavior for SAM against 7bPRMT7 (H4₍₁₋₂₀₎ sat.)—Experiments were conducted following matching CePRMT5 protocol above. Briefly, the measurements consisted of two experimental subsets: MTase reactions (both SAM and peptide added) and MTase blanks (SAM was added without peptide to account for natural SAM decomposition). The 250-μL reaction mixtures from a same row contained 25 mM phosphate buffer pH=7.60, 1 mM βME, 10% glycerol, 4 μM coupling deaminase TM0936, 402 nM TbPRMT7 and various SAM concentrations (1.53-20.0 μM). Absorbance at 263 nm was recorded after addition of either H4₍₁₋₂₀₎ (200 μM saturating final concentration; MTase reactions) or water (MTase blanks). Initial rates were first corrected for background signal, then plotted against SAM concentrations and fitted to the Morrison kinetic model to yield corresponding kinetic parameters.

Kinetic behavior for peptide H3₍₁₋₅₃₎ against NcDIM-S(SAM sat.)—Experiments were conducted following matching CePRMT5 protocol above. Briefly, the 250-μL reaction mixtures from a same row contained 25 mM glycine buffer pH=9.50, 1 mM βME, 10% glycerol, 50 mM NaCl, 4 μM coupling deaminase TM0936, 7.6 nM NcDIM-5 and various peptide concentrations (0.51-12.68 μM; in the first well, peptide acceptor was omitted to account for background signal—natural decomposition of SAM). Reactions started upon addition of SAM (25.8 μM saturating final concentration) and absorbance was recorded. Initial rates were first corrected for background signal, then plotted against H3₍₁₋₅₃₎ concentrations. Fit to the Michaelis-Menten equation provided corresponding kinetic parameters.

Kinetic behavior for sarcosine against GsSDMT (SAM sat.)—Experiments were conducted following matching CePRMT5 protocol above. Briefly, the 100-μL mixtures from a same row contained 100 mM phosphate buffer pH=7.80, 1 mM βME, 5% glycerol, 100 μM MgCl₂, 4 μM coupling deaminase TM0936, 976 nM GsSDMT and various sarcosine concentrations (0.5-12.5 mM; in the first well, sarcosine was omitted to account for background signal—natural decomposition of SAM). Reactions started upon addition of SAM (750 μM saturating final concentration), 60-μL volumes were transferred onto UV plate and absorbance was recorded. Initial rates were first corrected for background signal, and then plotted against sarcosine concentrations. Fit to the Michaelis-Menten equation provided corresponding kinetic parameters.

Kinetic behavior for SAM against GsSDMT (sarcosine sat.)—Experiments were conducted following matching CePRMT5 protocol above. Briefly, the measurements consisted of two experimental subsets: MTase reactions (both SAM and sarcosine added) and MTase blanks (SAM was added without sarcosine to account for natural SAM decomposition). The 100-μL reaction mixtures from a same row contained 100 mM phosphate buffer pH=7.80, 1 mM βME, 5% glycerol, 100 μM MgCl₂, 4 μM coupling deaminase TM0936, 195 nM GsSDMT and various SAM concentrations (49.9-750.2 μM). Reactions started upon addition of either sarcosine (10 mM saturating final concentration; MTase reactions) or water (MTase blanks), 60-μL volumes were transferred onto UV plate and absorbance was recorded at 263 nm. Initial rates were first corrected for background signal, and then plotted against SAM concentrations. Fit to the Michaelis-Menten equation provided corresponding kinetic parameters.

Kinetic behavior for 8-aza-SAM against MTases (acceptor sat.)—Briefly, kinetic parameters were determined at 20° C. in a UV-Star 96-well flat bottom plate (Greiner Bio-One, #655801) by continuous monitoring of absorbance at 282 nm using a SpectraMax M5 instrument (Molecular Devices). As for SAM experiments, the measurements consisted of two experimental subsets: MTase reactions (both 8-aza-SAM and acceptor added) and MTase blanks (8-aza-SAM was added without acceptor to account for natural 8-aza-SAM decomposition). The 250-μL (60-μL) reaction mixtures from a same row contained 50 mM phosphate buffer pH=7.60 (7.80), 1 mM βME, 10% glycerol (5% glycerol, 100 μM MgCl₂), 4 μM coupling deaminase TM0936, either 1.5 μM CePRMT5 or 402 nM TbPRMT7 (or 610 nM GsSDMT) and various 8-aza-SAM concentrations. Absorbance at 282 nm was recorded after addition of either H4₍₁₋₂₀₎ (104 μM saturating final concentration; MTase reactions) or water (MTase blanks) (of either sarcosine (5 mM saturating final concentration; MTase reactions) or water (MTase blanks)). Initial rates were first corrected for background signal, then plotted against 8-aza-SAM concentrations and fitted to the Michaelis-Menten equation to yield corresponding kinetic parameters. The kinetic data from CePRMT5 were fit to the following Eq. 6 to reflect substrate inhibition observed at higher 8-aza-SAM concentration:

$$v = \frac{{k}_{cat} [ MTase ] [ 8 - aza - SAM ]}{{K}_{m} + [ 8 - aza - SAM ] + \frac{{[ 8 - aza - SAM ]}^{2}}{{K}_{s}}}$$

where [MTase] and [8-aza-SAM] are the total concentration of methyltransferase and methyl donor, respectively; Kₘ is the Michaelis constant, K_{f} is the substrate inhibition constant and k_{cat} is the turnover for 8-aza-SAM substrate. Please note that DIM-5 was unable to accept 8-aza-SAM substrate.

### Study of GsSDMT Inhibition by Sinefungin (UV-Mode)

Briefly, kinetic parameters were determined at 20° C. in a UV-Star 96-well flat bottom plate (Greiner Bio-One, #655801) by continuous monitoring of absorbance at 263 nm using a SpectraMax M5 instrument (Molecular Devices). The 100-μL reaction mixtures from a set of two rows contained 100 mM phosphate buffer pH=6.80, 1 mM βME, 5% glycerol, 100 AM MgCl₂, 4 μM coupling deaminase TM0936, 195 nM GsSDMT, saturating level of SAM (763 μM) and various sinefungin concentrations (0.8-244 μM). In the first row, the sarcosine acceptor was omitted to account for background signal (i.e. natural catabolism of sinefungin and SAM decomposition). The methyltransfer reactions started upon addition of sarcosine (5 mM saturating final concentration). Volumes (60-μL) were transferred onto plate and absorbance signals were recorded. The initial rates of the reactions were corrected using data from first row experiments and plotted against sinefungin concentrations. Fit to the following Eq. 7 provided corresponding inhibition constant (Kᵢ):

$$\frac{V}{{V}_{0}} = \frac{{K}_{m} + [ SAM ]}{{K}_{m} + [ SAM ] + ( {{K}_{m} ( \frac{{10}^{Log [ SIN ]}}{{K}_{i}} )}^{γ} )}$$

where V and V₀ are the initial velocity with and without inhibitor, respectively. The Michelis constant for SAM substrate is depicted as Kₘ and [SAM] is the concentration of this same molecule. The inhibition constant for sinefungin is represented by Kᵢ and [SIN] is the concentration of this inhibitor. The parameter γ is the Hill coefficient.
The Kcat and Kcat/Km pH-Dependence for the GsSDMT Reaction

Experiments were conducted following GsSDMT protocol above using 100 mM buffer (pH 5.80-9.88), 1 mM βME, 5% glycerol, 100 μM MgCl₂, 4 μM coupling deaminase TM0936, 976 nM GsSDMT and various sarcosine concentrations (0.5-12.5 mM; in the first well, sarcosine was omitted to account for background signal—natural decomposition of SAM). Reactions started upon addition of SAM (750 μM saturating final concentration) and 60-μL volumes were transferred onto UV-plate. Absorbance at 263 nm was recorded. After correction for background signal, initial velocities were plotted against sarcosine concentrations. Fit to the Michaelis-Menten equation provided corresponding Kₘ and k_{cat}. Both kinetic parameters were used to plot the corresponding pHdependences using the two following Eq. 8 and Eq. 9 (85):

$$log ( \frac{{k}_{cat}}{{K}_{m}} ) = log ( {V}_{max}^{MAX} ) - log ( {( 1 + \frac{{10}^{- pH}}{{10}^{- {pK}_{a}^{1}}} + \frac{{10}^{- {pK}_{a}^{2}}}{{10}^{- pH}} )}^{2} )$$

where Km and kcat are the Michaelis constant and turnover value, respectively, for sarcosine substrate at saturating levels of SAM (750 μM). Vₘₐₓ^{MAX} is the maximum velocity ever achieved by the GsSDMT enzyme over the full pH-range. The −pKₐ¹ and −pKₐ² are the logarithm of acid dissociation constant for a first and a second ionizable group of important entities: these entities are free sarcosine and GsSDMT•SAM complex.

$$log ( {k}_{cat} ) = log ( {k}_{cat}^{MAX} ) - log ( 1 + \frac{{10}^{- pH}}{{10}^{- {pK}_{a}^{1}}} )$$

where k_{cat} is turnover value for sarcosine substrate at saturating levels of SAM (750 μM); k_{cat}^{MAX} is maximum turnover value for sarcosine substrate ever reached; the parameter −pKₐ1 is the logarithm of the acid dissociation constant from a crucial ionizable group onto the GsSDMT•SAM•sarcosine complex.
Impact of Ionic Strength onto Km for H4₍₁₋₂₀₎ (TbPRMT7)

Experiments were conducted following TbPRMT7 protocol above. Briefly, the 250-μL reaction mixtures from a same row contained either 25, 50, 75 or 100 mM phosphate buffer pH=7.60, 1 mM βME, 10% glycerol, 4 μM coupling deaminase TM0936, 1.0 μM TbPRMT7 and various peptide concentrations (16.6-243.3 μM; in the first well, peptide acceptor was omitted to account for background signal—natural decomposition of SAM). Reactions started upon addition of SAM (25.8 μM saturating final concentration) and absorbance was recorded. Initial rates were first corrected for background signal, then plotted against H4₍₁₋₂₀₎ concentrations. The four fits to the Michaelis-Menten equation (k_{cat} fixed at 29 h⁻¹) provided corresponding Kₘ values and established impact of ionic strength onto this kinetic parameter.

### Fluorescence-Based Coupled Assay

Calibration of fluorescence signal for the 8-aza-A to 8-aza-I reaction—This procedure is very similar to the one described under “Differential Extinction Coefficients (Δε) for Deaminase Reaction”. Briefly, standard solutions (240 μL) of 8-aza-adenosine (0-25 μM; 12 data points) containing 50 mM buffer (pH 5.00-9.50), 10% glycerol and 1 mM βME, were loaded into a %-well black flat bottom plate. 10-μL water per well were added to one data-set (12 concentrations of 8-aza-adensoine); 10-μL TM0936 solution (24.05 μM) were added to the remaining samples (12 concentrations of 8-azaadenosine at various pH values). The loss of fluorescence (Em=360 nm) from 8-azaadenosine standard solutions (Ex=282/292 nm) where recorded with a Spectramax M5 plate reader until no change in signal could be observed.

At a same pH, the comparison between end-point deaminase reactions and reference well (TM0936 added but no 8-aza-adenosine) provided the pH-dependence of 8-azainosyl fluorescence. Using the Ex/Em pair (282/360 nm), the data fit to the Eq. 2 provided the following parameters: FLUO_{LOW pH}=0 RLU μM⁻¹, FLUO_{HIGH pH}=35.0±0.5 RLU μM⁻¹, pKₐ=7.50±0.03. Likewise, using the Ex/Em pair (292/360 mu), the data fit to the Eq. 2 provided the following parameters: FLUO_{LOW pH}=1.5±0.3 RLU μM⁻¹, FLUO_{HIGH pH}=22.0±0.3 RLU μM⁻¹, pKₐ=7.54±0.04. Furthermore, the comparison between end-point deaminase reactions and reference well (various 8-aza-adenosine concentrations but no TM0936 added) provided the calibration curve of fluorescence signal for the 8-aza-adenosine to 8-aza-ionisine reaction. Using the Ex/Em pairs (282/360 and 292/360 mu), the data fit to the Eq. 10 provided the calibration parameters. ΔFLUO=a[8-aza-A]²+b[8-aza-A]+c These parameters are summarized in Table 2.

Kinetic behavior for H4₍₁₋₂₀₎ against TbPRM77 using 8-aza-SAM—Experiments were conducted following TbPRMT7 protocol above. Briefly, the 250-μL reaction mixtures from a same row contained 25 mM phosphate buffer pH=7.60, 1 mM βME, 10% glycerol, 4 μM coupling deaminase TM0936, 805.2 nM TbPRMT7 and various peptide concentrations (0-144.55 μM. The first well contained only 8-aza-SAM without any coupling enzyme, nor methyltransferase; in the second well, peptide acceptor was omitted to account for background signal—natural decomposition of 8-aza-SAM). Reactions started upon addition of cofactor (25.0 μM saturating final concentration) and absorbance was recorded. Initial rates were first corrected for background signal, then plotted against H4₍₁₋₂₀₎ concentrations. The data fit to the Michaelis-Menten equation S5 provided corresponding k_{cat} of 39±2h⁻¹ and a Km value of 71±8 μM.

### Results and Discussion

The deaminase TM0936 is a prime-choice candidate for MTase assays. We used a two-step purification of TM0936 as a polyhistidine-tagged protein expressed in bacteria. The high-purity enzyme from the hyperthermophile Thermotoga maritima can sustain multiple freeze/thaw cycles, it remains unable to catabolize SAM and it displays an excellent reactivity towards SAH at room temperature (Kₘ=0.6±0.2 μM, k_{cat}=7.1±0.4 s⁻¹) (41,43).

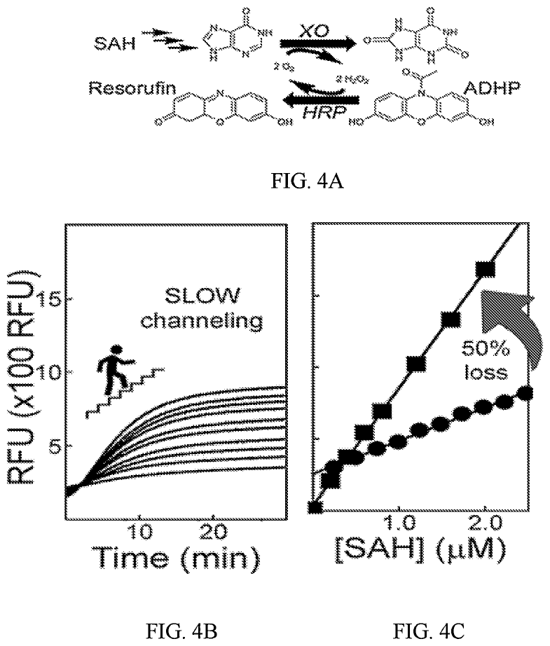

Coupled-enzyme assays for MTase reactions are based on the same principal of rapid channeling of SAH to a signal output so that SAH is virtually absent and coupling enzymes are not rate-limiting. Thus, the signal output solely reflects the MTase reaction. In our hands, commercial kits for detection of methyl transfer (FIG. 4A; Cayman Chemical, #700150) suffered from poor performances with a slow and incomplete processing of SAH. It took 10 min for a 200-nM standard concentration of SAH to be digested by coupling enzymes (FIG. 4B). Furthermore, a comparison between SAH and resorufin standard curves supports that channeling of the MTase product is incomplete, thus resulting in a 50% loss of sensitivity (FIG. 4C).

We previously developed methods for detection of SAH and its derivatives (e.g. adenosine, adenine) (37,45). Although we routinely use these highly sensitive luciferase-based coupled assays (46), we encountered limitations. The preparation of four highly purified recombinant enzymes (i.e. MTAN, APRT, PPDK, FLUC) is a tedious process and batch-to-batch reproducibility may be an issue. Furthermore, the coupling buffer requires 5-phospho-a-D-ribosyl-1-pyrophosphate (PRPP), a highly unstable and expensive substrate to fuel APRT. Therefore, based on the TM0936 performance, we developed the 1-Step EZ-MTase assay to provide a simple and robust method for measuring SAM consumption.

Spectral signature of the SAH deamination reaction catalyzed by TM0936. To determine the precise relationship between absorbance and concentration for the adenosyl to inosyl conversion, we measured the differential extinction coefficient from the reaction catalyzed by TM0936. A nucleoside absorbance spectrum (i.e. maximum absorption wavelength λ max, extinction coefficient e) is pH-dependent, thus reflects the ionization state of this molecule. The adenosine UV-signature remains unchanged in water under most conditions (neutral species at pH>3.6); however, inosine may behave differently as it becomes deprotonated in alkaline solutions (pKₐ=8.9) (47,48).

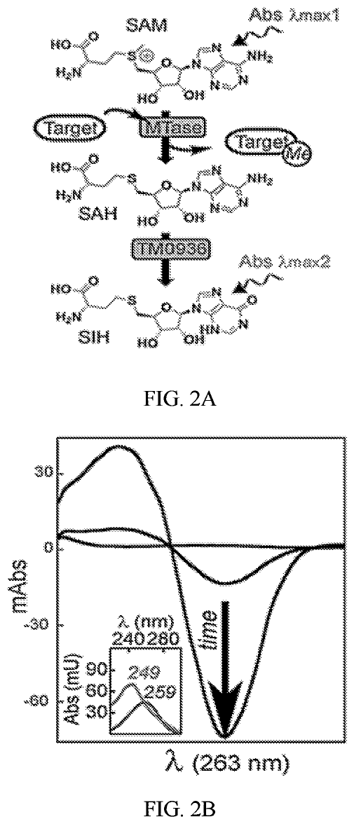

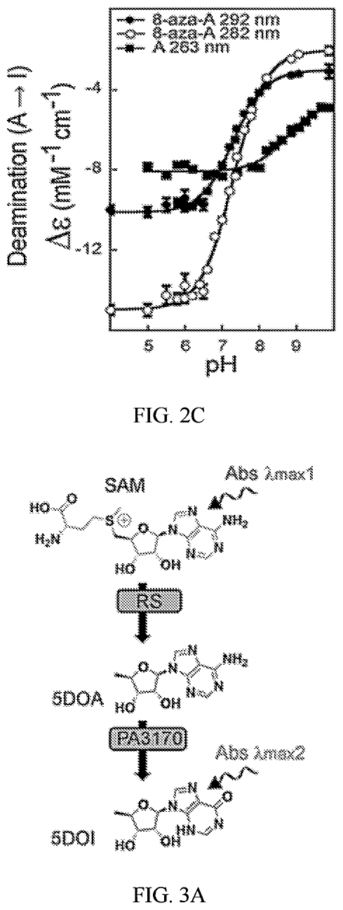

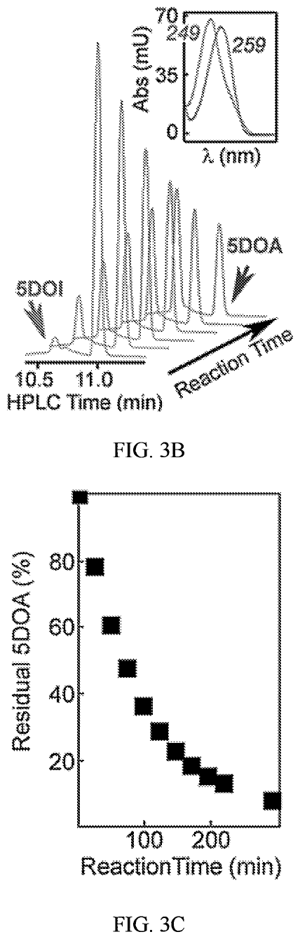

We established the Δε=f(pH) relationship for the deamination(FIG. 2C). This data-set is a key component of the assay so that MTase reaction rates may be determined accurately across a broad pH-range. Data were fitted to Eq. 2 where Δε_{HIGH pH} (−4,655±186 M⁻¹ cm⁻¹) and Δε_{LOW pH} (−8,076±80 M⁻¹ cm⁻¹) are the extinction coefficient measured at 263 nm for the deaminase reaction at high and low pH, respectively, and −pKₐ (−8.72±0.09) is the logarithm of acid dissociation constant for inosine (lit. (49), −3,890 M⁻¹ cm⁻¹, −8,270 M⁻¹ cm⁻¹ and 8.85, respectively).

In addition, similar Δε=f(pH) relationships were established at 282 and 292 nm for the 8-aza-adenosine (8-aza-A) to 8-aza-inosine (8-aza-I) reaction (FIG. 2C). The 8aza analogs of SAM/SAH are fluorescent while their inosyl counterparts are not (50,51). Thus, 8-aza-SAM may be a valuable substrate for monitoring MTase activity using a fluorescence mode. Our experiments provided: Δε_{HIGH pH} (−2,026±174 and −3,016±119 M⁻¹ cm⁻¹), Δε_{LOW pH} (−14,975±129 and −10,117±87 M⁻¹ cm⁻¹) and pKₐ (7.29±0.03 and 7.30±0.03), at 282 and 292 nm, respectively.

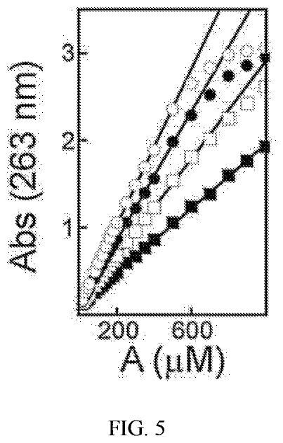

Kinetic parameters from four methyltransferases using 1-Step EZ-MTase. To establish a coupled assay that can sample the vast majority of MTase targets, all having diverse kinetic properties (range of SAM concentrations: 1-1000 mM; FIG. 5), we performed experiments in 96-well plates compatible with UV detection. This format allows for higher throughput of data vs. a regular cell-changer spectrophotometer using ½-3 mL cuvettes. The wells accommodate 50-250 μL sample volumes, resulting in an adjustable optical path so that a linear relationship between absorbance and concentration is preserved (FIG. 5).

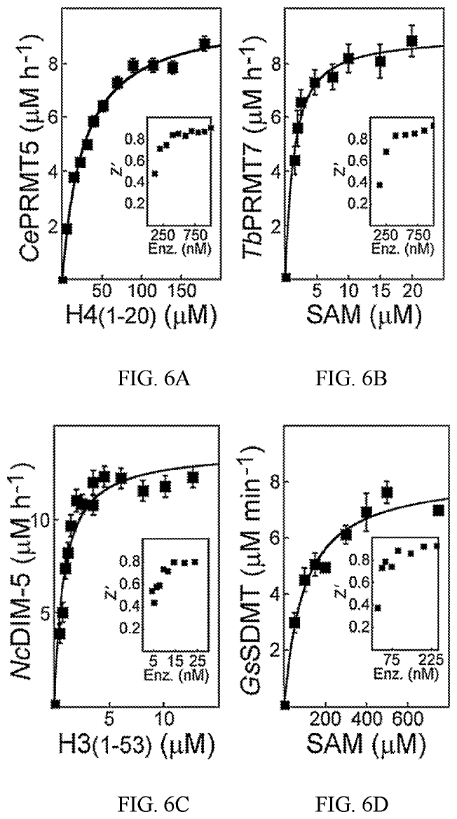

We tested this coupled assay with four unique methyltransferases. TM0936 (4 μM) was coupled to these transferases to establish their kinetic behavior: determination of Michaelis constant (Kₘ) and catalytic turnover (k_{cat}) for either SAM or acceptor substrate. Using the protein arginine methyltransferase from Caenorhabditis elegans (CePRMT5) and saturating levels of SAM (25 μM), we determined the kinetic profile as a function of the peptide H4₍₁₋₂₀₎ concentration (6-180 μM, at 300 nM CePRMT5). The data fit to the Morrison equation (52) gives a Kₘ of 26±2 μM and a k_{cat} of 32.9±0.8 h⁻¹ (FIG. 6A), consistent with values we reported using the luciferase-based assay (lit. (46), Kₘ=54±4 μM, k_{cat}=28.6±0.7 h⁻¹). Another member of the PRMT family, PRMT7 from Trypanosoma brucei (400 nM), was also tested using saturating concentration of peptide H4₍₁₋₂₀₎ (200 PM). Varying SAM concentration (1.5-20.0 μM), reaction rates were determined and plotted against cofactor concentration. The data were fitted as for CePRMT5, giving a Kₘ of 1.1±0.2 μM and a turnover of 22.3±0.6 h⁻¹. Likewise, the activity from Neurospora crassa protein lysine N-methyltransferase (histone H3 lysine-9 specific; NcDIM-5) was successfully detected at pH=9.50. Our data (Kₘ=0.9±0.1 μM, k_{cat}=30±1 min⁻¹) and reported values (lit. (53), Kₘ=7.4 μM, k_{cat}=138 h⁻¹) display a 100-fold difference in the overall catalytic efficiencies (2,000 μM⁻¹ h⁻¹ vs. 19 μM⁻¹ h⁻¹, respectively). However, we used a H3₍₁₋₅₃₎ peptide while previous report mentioned a H3₍₁₋₂₀₎ peptide harboring a N-terminal biotinylation; such a modification, vicinal to the reactive lysine-9, may account for the discrepancy observed. Finally, we selected sarcosine/dimethylglycine N-methyltransferase from Galdieria sulphuraria (GsSDMT) to represent members of the SMMT family. We thought the 1-Step EZ-MTase assay would be challenged by this enzyme. Indeed, GsSDMT behaves differently from other MTases we tested: it is a fast enzyme and displays a high micromolar Kₘ for SAM (lit. (54), Kₘ=144±44 M, k_{cat}=52±4 min⁻¹). Yet, using a 60-mL reaction volume to accommodate the elevated SAM levels (50-750 μM), along with a low enzyme concentration (195 nM), we successfully determined the GsSDMT kinetic parameters. Both the Kₘ of 95±18 μM and the k_{cat} of 42±2 min¹ are in agreement with the data mentioned earlier. As we demonstrated with these four examples, the combination of a single coupling enzyme with a UV-mode of detection and a 96-well plate format makes the characterization of methyltransferases simple and straightforward. An overall summary of kinetic parameters from MTases we assayed is represented in Table 1.

TM0936 activity is resilient to pH variations. Another key limitation to the luciferase-based assays is their sensitivity in different chemical environments, as the detection is optimum at a very narrow pH value (≈7.7) and luminescence output is drastically reduced upon pH variations. As the pH decreases, so does the green component of the luminescence; light was no longer detected under acidic conditions (pH<6.0)(55). Therefore, key mechanistic insights are undetectable with this assay as it is impossible to describe MTase enzymology over a wide pH-range.

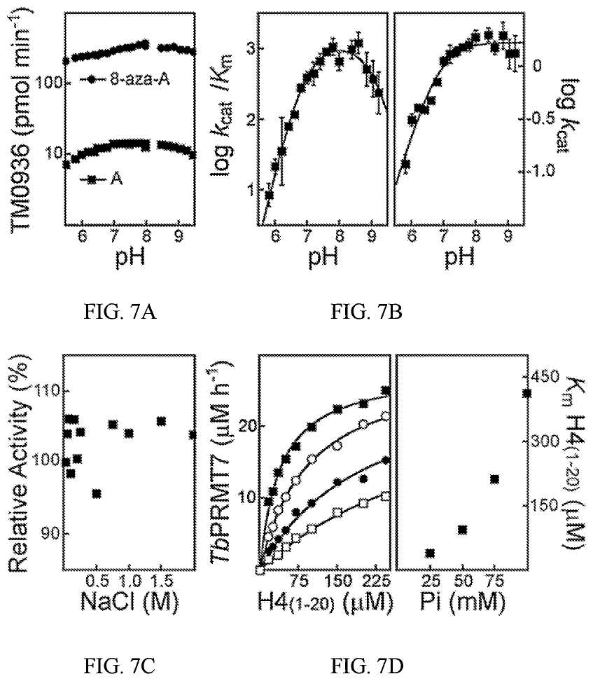

In contrast, TM0936 displays a sustained activity across a broad pH-range (FIG. 7A). With the 1-Step EZ-MTase assay, the transferase rates are the limiting ones at all pHs and absorbance recordings directly relate to methyl transfer. This is a characteristic to consider and take advantage of when developing a coupled enzymatic assay. As support of broad utility of this assay, we harnessed the deaminase activity of TM0936 to probe the enzymatic mechanism from a poorly characterized methyltransferase: sarcosine/dimethylglycine N-methyltransferase (SDMT). SDMT catalyzes a two-step methylation process leading to a key metabolite: betaine. Trimethylglycine is an effective methyl donor involved in the biosynthesis of L-methionine from L-homocysteine (56); furthermore, under extreme conditions (e.g. high salt concentrations or low temperatures), this molecule stabilizes proteins acting as an osmoprotectant (57). Little mechanistic information is available regarding this enzyme, with a handful of kinetic reports (54, 58, 59) and one single crystal structure of the apo-form of SDMT from Galdieria sulphuraria (54,60). In an experimental tour de force, we established the pH-dependence of GsSDMT reaction rates for sarcosine (Dixon plots; FIG. 7B). By reporting Kₘ and k_{cat} for this substrate across a 4-unit range of pHs, we gleaned valuable information and proposed an inventory of residues potentially involved in sarcosine capture and processing by the SDMT enzyme.

Fitted to the Eq. 8, the log k_{cat}/Kₘ vs. pH displays a symmetrical bell-shaped curve with an optimum enzymatic activity between pH 7.5-8.5; pKₐ values of 6.87±0.05 and 9.2±0.1 were assigned to the ascending and descending limbs, respectively (FIG. 7B, left). Since sarcosine was the varied substrate, the k_{cat}/Kₘ is the apparent second-order rate constant for the reaction between free sarcosine and GsSDMT•SAM complex. Thus, the effects of pH onto this rate constant likely describe the ionization states of these two entities. The protonation of sarcosine carboxylate (pKₐ≈2.2) does not explain the loss of activity at acidic pHs. Indeed, the pKₐ for sarcosine carboxylate is much lower than the observed 6.87 pKₐ-value for the ascending limb. Such a pKₐ-value may be reminiscent of histidine residues (pKₐ 6.0-7.0) important for efficient sequestration of sarcosine by the GsSDMT•SAM complex. With respect to the descending limb (pKₐ=9.2), the deprotonation of either the methyl-amine group from sarcosine (pKₐ═10.0) or a tyrosine residue (pKₐ=10.1) may account for the loss of enzymatic activity under alkaline conditions.

The log k_{cat} plotted against pH (FIG. 7B, right; Eq. 9) established that reaction rates increased with pH and reached a maximum value at pH=6.82. This plot reports the ionization state of an important residue from the GsSDMT•SAM•sarcosine complex. Our results support our hypothesis that deprotonation of a crucial histidine may enhance the methyltransfer reaction.

One single structure of SDMT has been reported (54). We successfully superimposed this structure (PDB: 2O57) (54) with the SAH•sarcosine complex of the glycine sarcosine N-methyltransferase from Methanohalophilus portucalensis (MpGSMT; PDB: 5HIL) (61). Both structures display a good alignment of their N-terminus and the cofactor binding site was easily identified within this conserved domain. Indeed, SAM interacts with key conserved amino-acids: π-stacking between adenosine and W115, stabilization of the ribosyl through hydrogen bond with D88 (F141 and N112 from GsSDMT are predicted to be homologous). Likewise, the homocysteyl binding-mode depicted additional groups involved in stabilization of the cofactor. Residues R60, A91 and Q157 from GsSDMT are structurally homologous to R43, A67 and L132 from MpGSMT, respectively; thus we hypothesize their interaction with the homocysteyl moiety from SAM/SAH. Although the sarcosine binding site is located within the most divergent region of the structures (C-terminus), we identified Y242 (Y206 structural homolog in MpGSMT) and H241 from GsSDMT as potential candidates involved in sarcosine stabilization via hydrogen bond with the carboxylate tail. Furthermore, the histidine H162 from GsSDMT, also present in MpGSMT (H138), may influence methyl transfer rate since it is equidistant from both nitrogen and sulfur reactive centers from sarcosine and SAH, respectively.

TM0936 activity is resilient to variations of ionic strength. We previously established the role of the MEP50 WD-repeat protein in presenting histone substrates to the PRMT5 active site (46). To measure the sub-micromolar affinity between MEP50 and H4₍₁₋₂₀₎ peptide, we relieved methyl transfer activity through titration of exogenous MEP50. Many WD-repeat proteins are highly hydrophobic and require high salt concentrations to promote their solubility (62). Although we had monitored methyl transfer with the highly sensitive luciferase coupled assay, it was critical to maintain ionic strength constant throughout MEP50 titration (46). Indeed, a slight increasing in salt concentration drastically decreases FLUC light output, making FLUC-based assays more difficult. Importantly, TM0936 was not affected by increasing levels of salt. In our hands, the deaminase steadily catabolized adenosine (10 AM), even at sodium chloride concentrations as high as 2M (FIG. 7C).

To test the utility of this new assay in varied ionic conditions, we measured histone methyltransferase activity. Histone tails are positively charged under physiological conditions, as they are enriched in lysine and arginine residues; this electrostatic property may partially account for binding of these substrates onto MTase target. Using TbPRMT7, we determined the kinetic behavior for H4₍₁₋₂₀₎ peptide at four phosphate concentrations (pH=7.60, 25-100 mM). The initial rates were plotted against peptide concentrations (FIG. 7D, left). The data fit to the Michaelis-Menten equation (Eq. 5) gives four Kₘ values (AM) of 39±3, 93±5, 212±7, 413±8 at 25, 50, 75 and 100 mM phosphate concentrations, respectively (FIG. 7D, right). As the buffer concentration increased, so did the Kₘ value for the histone peptide, thus reflecting a loss of affinity between substrate and PRMT7. Our data are in good agreement with the isothermal titration calorimetry experiments performed with the same PRMT7/peptide pair. While a 20-fold decrease in affinity between peptide and PRMT7 was observed when varying salt concentration from 20 to 150 mM, a 300 mM salt concentration precluded binding event (lit. (63), K_{d}≈400,000 μM).

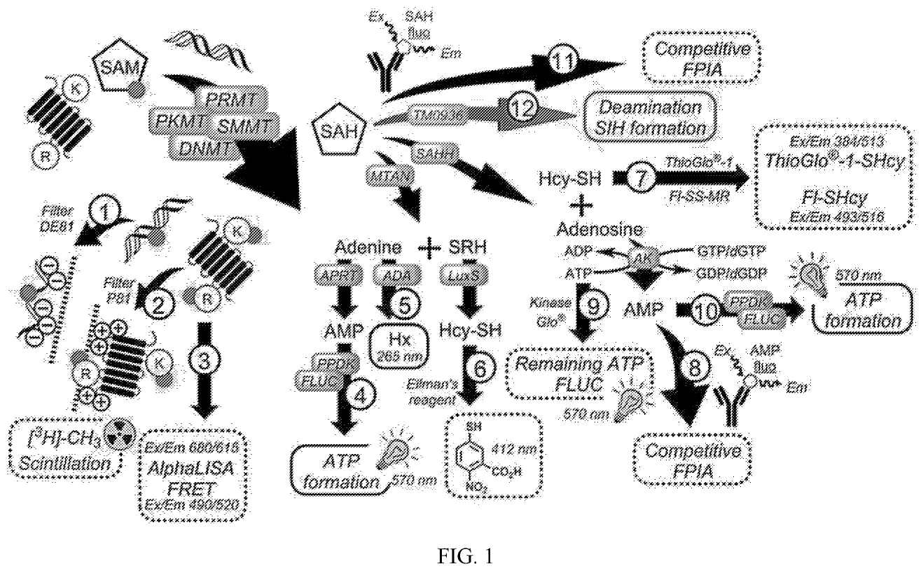

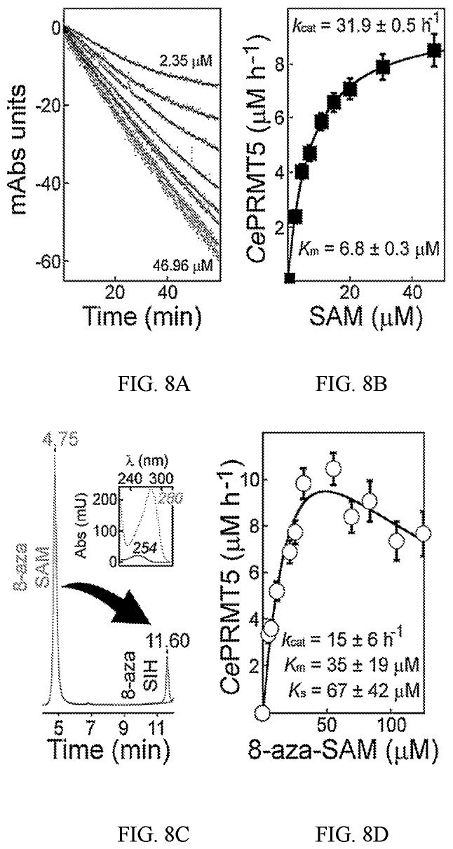

1-Step EZ-MTase is well suited for high-throughput screening. Our analytical tool provides an alternative to overcome major drawbacks from previous MTase assays (FIG. 1); methods involved as much as four coupling enzymes, and the single deaminase activity from 1-Step EZ-MTase may decrease the risk for off-target inhibition and apparition of false positives. A high sensitivity and wide dynamic range of detection make this assay very competitive. Methyl transfer rates as low as 2 μM h⁻¹ are detectable and the use of nanomolar MTase concentrations is also achieved, important as many eukaryotic MTases are difficult to purify in quantity sufficient for other assays (FIGS. 8A and 8B). Very good values of screening window coefficient (Z′-factor) are obtained and always above 0.50, reaching maximum values of 0.87, 0.93, 0.80 and 0.92 for CePRMT5, TbPRMT7, NcDIM-5 and GsSDMT, respectively (FIG. 6; inserts) (64). Such high values reflect the overall quality of the 1-Step EZ-MTase assay. Our analytical tool is well suited for HTS, with a signal displaying low variability and a good separation from background (e.g. SAM decomposition).

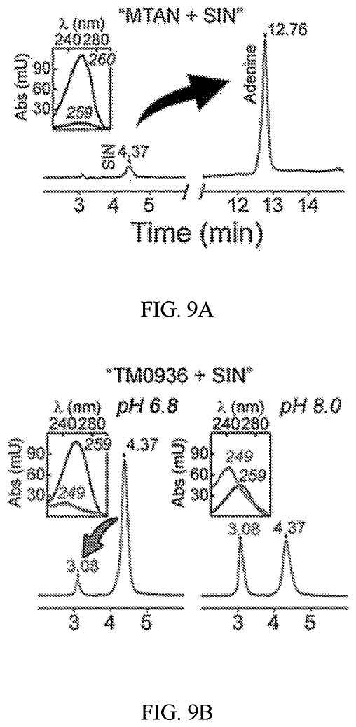

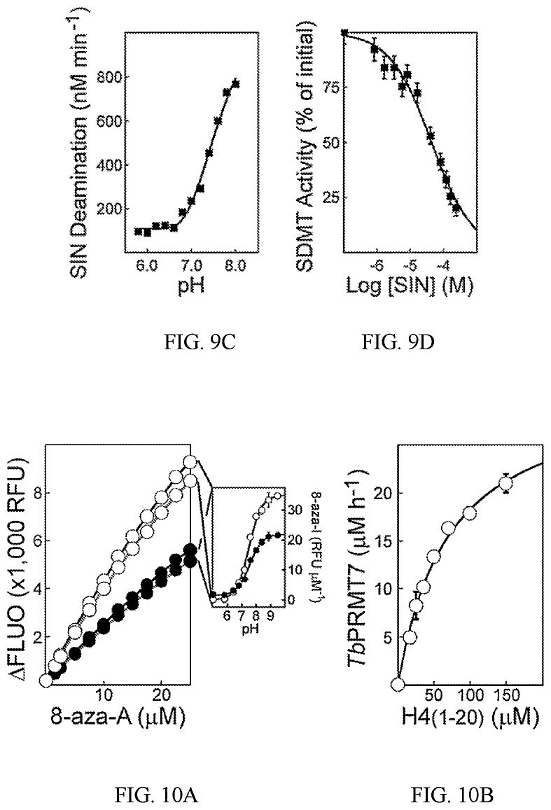

Sinefingin is a potent, yet non-selective inhibitor of MTAse; so it often serves as positive control during inhibitor screening. On one hand, sinefungin is a known inhibitor of SAHH enzyme (36,65); on the other hand, MTAN catabolizes sinefungin into adenine (FIG. 9A). Thus, most enzyme-coupled assays for MTase analysis are incompatible with this molecule (FIG. 1, arrows 4-10). We evaluated the possibility to use sinefungin within the 1-Step EZ-MTase assay and established the reactivity profile between TM0936 and this chemical. Unlike other coupling enzymes, TM0936 displayed moderate reactivity towards this inhibitor and deamination of sinefungin was slower at pH=6.80 than at pH=8.00 (FIG. 9B). Indeed, the coupling enzyme deaminates the inhibitor very slowly under acidic conditions (pH≤56.80, 125 nM min⁻¹; FIG. 9C). As pH increases, so does the rate of deamination. The —NH₃⁺ group from sinefungin is deprotonated into —NH₂, thus becoming a lesser mimic of the CH₃—S⁺ group from SAM (pH=8.00, 760 nM min⁻¹; FIG. 9C). This observation is in good agreement with TM0936 inability to catabolize SAM.

Taking advantage of this substrate selectivity, we measured (pH=6.80) the inhibition constant (Kᵢ) for sinefungin against GsSDMT (FIG. 9D). Under our experimental conditions, sinefungin was a potent inhibitor of the reaction between sarcosine and SAM catalyzed by GsSDMT (K, =1.8±0.4 μM). This result demonstrated the compatibility of our enzyme-coupled assay with sinefungin.

8-aza-SAM and its application to the 1-Step EZ-MTase assay. A second mode of detection, complementary to the current UV-based assay, may present advantages. The use of a fluorescent cofactor analog may improve detection specificity compared with UV absorption. 8-aza-adenosine is an isosteric and fluorescent analog of the nucleoside. When excited at 282 nm, this probe exhibits a strong fluorescence signature with a maximum at 360 nm (50). This characteristic is not shared with the 8-aza-inosine and this deaminated product is a weak fluorophore (50). Several SAM analogs, including the 8-aza modification, are biologically active and display affinity with the SAM-III riboswitch, the EcoRI methyltransferase and other MTases (44,66). Therefore, we anticipated this probe may be used in place of the natural cofactor.

We successfully resynthesized 8-aza-SAM through reaction between 8-aza-ATP and L-methionine (66). SAM isomerizes readily at the sulfonium center and only the S(S)-epimer is biologically active. Thus, with performed a short reaction at 35° C. (enzyme:triphosphate, 1:400) to limit such epimerization and yield 8-aza-SAM (65% yield, 5% S(R)-epimer; cf. NMR analysis). Although 8-aza-ATP was commercially available (TriLink Biotechnologies, #N-1004), this molecule is extremely expensive; thus, we synthesize this chemical through multiple phosphorylation of the more affordable 8-aza-adenosine. This approach is reminiscent of a strategy we previously applied towards the preparation of a C-P lyase inhibitor (67). Performed on a 50-mg scale and in a 2-mL tube, this one-pot synthesis provides an easy access to high quantities of pure 8-aza-ATP (95%; cf. Enzymatic Syntheses).

We further assayed the reactivity of 8-aza-SAM towards our methyltransferases. This analog was well tolerated by our candidates since only NcDIM-5 was unable to process this cofactor. Likewise, the 8-aza-SAH product of methyltransfer reactions (λmax=280 nm) was successfully converted into the deaminated 8-aza-SIH (λmax=254 nm; FIG. 8C). In fact, the deamination of adenosine by TM0936 is 10-fold slower than that of its 8-aza analog (FIG. 7A). Using the UV-based assay, we determined the 8-aza-SAM kinetic parameters with CePRMT5 at saturating concentration of H4 peptide (FIG. 8D). Turnover was moderately affected and the enzyme processed SAM twice as fast as its 8-aza-analog (k_{cat}=31.9±0.5 h⁻¹ vs. 15±6 h⁻¹, respectively; FIGS. 8B and 8D). The catalytic efficiency for 8-aza-SAM was one order of magnitude lower than that of the natural cofactor (120 M⁻¹ s⁻¹ and 1300 M⁻¹ s⁻¹, respectively). Unlike SAM, the 8-aza analog displayed significant substrate inhibition (K_{f}=67±42 μM; FIG. 8D). When assayed against either TbPRMT7 or GsSDMT, this inhibitory behavior of the analog was not observed (Table 1).

With the relevance of 8-aza-SAM validated, we then calibrated the fluorescence signal at 360 nm for the 8-aza-adenosine (8-aza-A) to 8-aza-inosine (8-aza-I) reaction using two excitation wavelengths (i.e. 282 and 292 nm). The 282 nm excitation provided a higher limit of detection for 8-aza-A and fluorescence signal was most intense (≈2-fold; FIG. 10A). Plotted against 8-aza-A concentrations (0-25 μM), the variation of fluorescence (DFLUO) observed over deaminase reaction fits a polynomial equation (Eq. 10 and Table 2). Furthermore, experiments performed at various pHs (5.00-9.50) support the weak fluorescence properties of 8-aza-I. The deaminated product is a poor fluorophore, it does not emit light under acidic conditions and its fluorescence is 10-fold weaker than that of 8-aza-A at pH=9.50 (35 RLU mM⁻¹ vs. 380 RLU mM⁻¹; FIG. 10A, insert). Thus, at the early stage of methyltransfer reaction (i.e. less than 10% of 8-aza-SAM consumed), the 8-aza-SIH contribution to the fluorescence is negligible and accounts for less than 1% of the signal. To establish the utility of a fluorescence mode of detection, we measured kinetic parameters for the transfer reaction catalyzed by TbPRMT7 using 8-aza-SAM and various concentration of H4₍₁₋₂₀₎ peptide (FIG. 10B). The data fit to the Michaelis-Menten equation (Eq. 5) gives Kₘ and k_{cat} values of 71±8 μM and 39±2 h⁻¹, respectively (FIG. 10B).

Glycine N-methyltransferase (GNMT) is a key component of SAM homeostasis. As a methionine-rich diet replenishes the SAM pool, the increasing concentration of methyl donor inhibits the 5,10-methylene-tetrahydrofolate reductase, thus impairing the 5-methyltetrahydrofolate (5-CH₃-THF) synthesis. GNMT is a folate-binding protein and 5-CH₃-THF is deleterious to its activity. Within this feedback mechanism, the alleviating GNMT inhibition promotes SAM consumption through sarcosine synthesis. GNMT establishes the cross talk between the one carbon folate pathway and the methionine cycle, thereby maintaining a healthy SAM/SAH ratio, which is indicative of methylator potential.

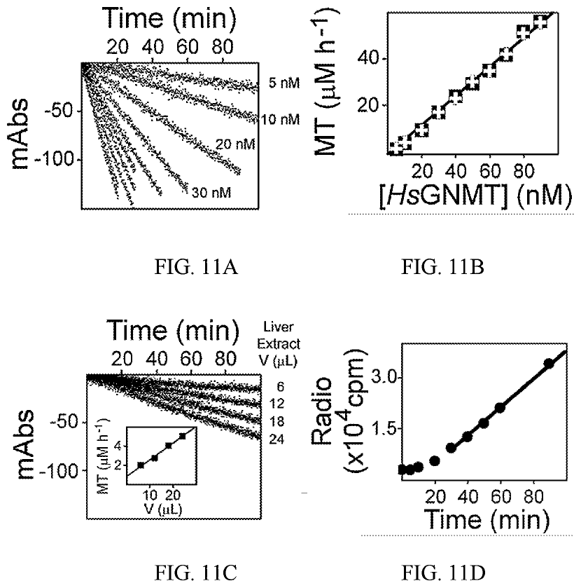

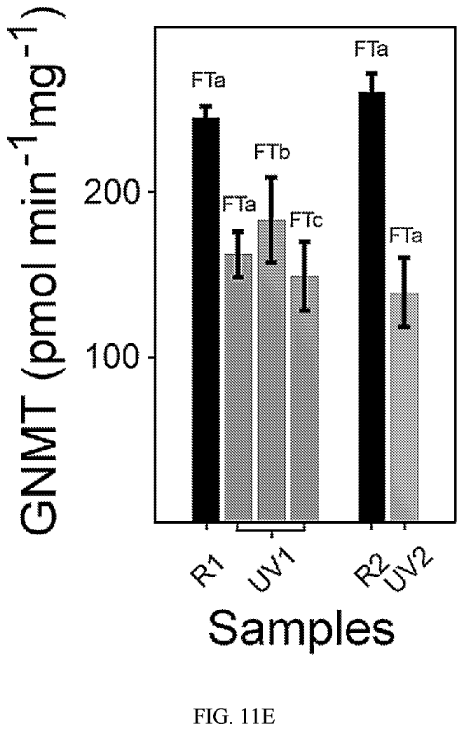

Development of improved assays for probing GNMT activity within biological samples is important to accelerate understanding of the interplay among metabolic pathways, energetics, epigenetics and cancer metabolism. However, classical methods using tritiated SAM, and the detection of radioactive sarcosine within crude protein extracts is a tedious and discontinuous approach. Furthermore, valuable tissue samples from animal studies may be limited and the requirement for large amounts of extract (e.g. 250 μg total protein) may be challenging when using this method. To overcome these drawbacks, we optimized the 1-Step EZ-MTase platform and made it compatible with biological samples. Using protein extracts from rat liver, we successfully quantitated GNMT activity within these crude biological samples (FIG. 11).

The deamination reaction catalyzed by TM0936 only occurs with SAH, methylthioadenosine and adenosine. The substrate specificity of our coupling enzyme makes it compatible with the highly complex content of biological samples. Luciferase-based assays are not suited for such a type of sample where adenine and phosphorylated adenosine species generate high background signal. Likewise, endogenous thiol species (e.g. glutathione, homocysteine, cysteine residues from proteins) preclude the continuous detection of GNMT activity through efficient derivatization of homocysteine (e.g. Ellman's reagent, ThioGlo®-1). In addition to reacting with these thiol species, the 5,5′-dithio-bis-(2-nitrobenzoic acid) completely inactivates the methyltransferase upon reaction with its cysteine residues.

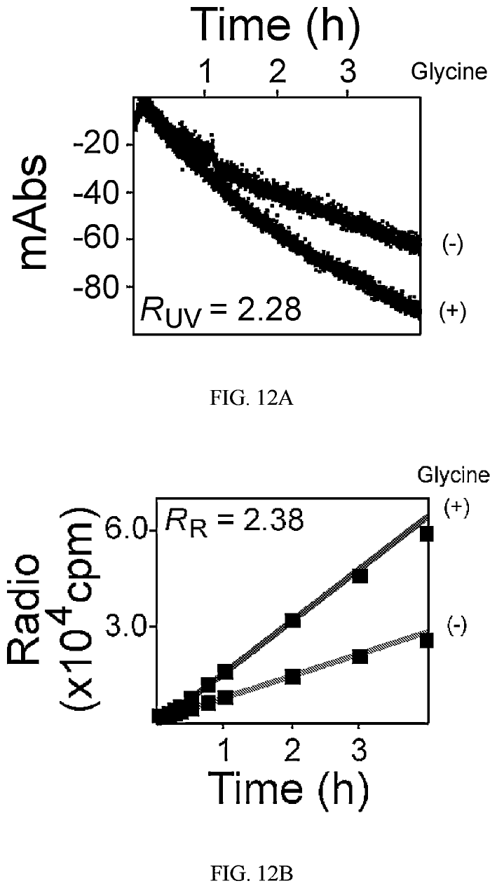

In our hand, GNMT activity was not affected by the tissue lysis buffer (150 mM NaCl, 20 mM Tris-HCl pH=7.4, 1% Triton X-100, 1 mM orthovanadate, 1 mM EDTA, 10 mM NaF, 1× protease inhibitor cocktail, and 1 mM PMSF), thus establishing the compatibility between this universal reagent and the 1-Step EZ-MTase (FIGS. 11A and 11B). With its low 2 μM h⁻¹ SAH-detection limit, our analytical tool successfully senses methyltransfer catalyzed by GNMT. At 75 μM SAM concentration, endogenous glycine was not sufficient for sarcosine synthesis catalyzed by rat GNMT (FIG. 12). Upon addition of saturating glycine (20 mM), a 2.28-fold increase of methyltransfer rate was observed (FIG. 12A; 2.38-fold increase as monitored with the radioactive assay, FIG. 12B). Our observations are in good agreement with the estimated 50-100 μM endogenous glycine concentration (2.37 mM in rat liver) and a Kₘ value for glycine of 130 μM.

The decrease of absorbance at 263 nm is concentration dependent and levels as low as 30 μg of total protein were sufficient to monitor sarcosine synthesis over an extended period (FIG. 11C). Our platform surpasses the performance of the tedious radioactive assay (FIG. 11D) by only requiring a fraction of the material necessary for the radioactive assay, and yet remains capable of continuously detecting the SAH methyltransferase product. Values obtained with both methods were comparable (radio assay and spectro-based assay, R and UV, respectively; FIG. 11E) and GNMT displayed similar activity across liver samples (162±14 and 139±21 pmol min⁻¹ mg⁻¹ for UV1 and UV2, respectively; 245±7 and 261±11 pmol min⁻¹ mg⁻¹ for R1 and R2, respectively). To evaluate the GNMT stability within liver extracts, we measured GNMT activity after three freeze-thaw cycles (FTa-FTc; FIG. 11E). Our results display overlapping measurements (162±14, 183±26 and 149±21 pmol min⁻¹ mg⁻¹ for FTa, FTb and FTc, respectively; FIG. 11E), confirming that GNMT enzyme activity is very stable against multiple freeze-thaw cycles.

### Conclusion

Epigenetic modifications catalyzed by methyltransferases play a central role in gene transcription and parental imprinting. A correlation between dysregulation of methylation patterns and occurrence of human diseases (e.g. cancer, diabetes) is becoming more obvious. Several methyltransferases are now validated therapeutic targets. The regulation of these enzymatic activities by specific and potent inhibitors may offer new opportunities for patients. To promote our understanding of methyltransferases and the discovery of new chemotherapeutics, we have developed a simple and straightforward assay to study this class of enzymes. Unlike anything else available, this analytical tool harnesses the power of one single protein: the SAH-deaminase TM0936. The coupling enzyme is easily accessible, it is also a powerful and sturdy catalyst that allows for quick and facile determination of enzymatic rates through monitoring of absorbance at 263 nm. We demonstrated its use by coupling it to a panel of four enzymes including lysine and arginine methyltransferases. A 96-well plate format allowed high-throughput performance of the assay. It detects transfer rates as low as 2 mM h⁻¹, accommodates nanomolar MTase concentration and display high Z′-factors, thus reflecting the overall quality of this assay. Furthermore, sinefungin is compatible with this coupled assay, so the tool may have a significant impact on the identification of new inhibitors using high throughput screening. We implemented a second mode of detection, providing users with the option to detect methyl transfer through monitoring the loss of fluorescence at 360 nm. This approach relies on 8-aza-SAM, a fluorescent analog of the universal methyl donor. We established the relevance of this alternative cofactor and determine kinetic properties of the PRMT7 from Trypanosoma brucei using H4₍₁₋₂₀₎ peptide.

TABLE 1
The kinetic parameters Kₘ and k_{cat} for several
methyltransferases, using SAM and 8-aza-SAM
as methyl donor and several methyl acceptors.

| | |
|---|---|
| Kₘ | K_{cat} |

CePRMT5

| | | | | |
|---|---|---|---|---|
| SAM | 6.8 ± 0.3 | μM | 31.9 ± 0.5 | h⁻¹ |
| H4₍₁₋₂₀₎ | 26 ± 2 | μM | 32.9 ± 0.8 | h⁻¹ |
| 8-aza-SAM | 35 ± 20 | μM | 15 ± 6 | h⁻¹ |

TbPRMT7

| | | | | |
|---|---|---|---|---|
| SAM | 1.1 ± 0.2 | μM | 22.3 ± 0.6 | h⁻¹ |
| H4₍₁₋₂₀₎ | 39 ± 3 | μM | 28.2 ± 0.7 | h⁻¹ |
| 8-aza-SAM | 15.3 ± 0.7 | μM | 23.0 ± 0.4 | h⁻¹ |

NcDIM-5

| | | | | |
|---|---|---|---|---|
| SAM^{a} | 0.68 ± 0.20 | μM | 3.1 | h⁻¹ |
| H3₍₁₋₅₃₎ | 0.9 ± 0.1 | μM | 30 ± 1 | min⁻¹ |

| | | |
|---|---|---|
| 8-aza-SAM | NOT A | NOT A |

GsSDMT

| | | | | |
|---|---|---|---|---|
| SAM | 95 ± 18 | μM | 42 ± 2 | min⁻¹ |
| Sarcosine | 1.7 ± 0.2 | μM | 90 ± 5 | min⁻¹ |
| 8-aza-SAM | 443 ± 33 | μM | 81 ± 3 | min⁻¹ |

^{a}From P. Rathert, X. Cheng and A. Jeltsch, BioTechniques, 2007, 43, 602, 604, 606.

TABLE 2
Parameters for data fit regarding calibration of fluorescence
signal for the 8-aza-adenosine to 8-aza-inosine reaction.
Experiments performed at pH = 5.00 and pH = 9.50.

| | ΔFLUO (RFU) = a [8-aza-A]² + b [8-aza-A] + c | | | |
|---|---|---|---|---|
| CURVES | Ex/Em 282/360 nm | | | |
| | pH = 5.00 | −3.4 ± 0.2 | 450 ± 4 | 185 |
| | pH = 9.50 | −3.2 ± 0.1 | 414 ± 2 | 185 |
| | Ex/Em 282/360 nm | | | |
| | pH = 5.00 | −1.3 ± 0.1 | 253 ± 3 | 118 |
| | pH = 9.50 | −1.32 ± 0.06 | 236 ± 1 | 118 |

### REFERENCES

1. F. Wold, Annual review of biochemistry, 1981, 50, 783-814.
2. T. Hitosugi and J. Chen, Oncogene, 2014, 33, 4279-4285.
3. A. P. Lothrop, M. P. Torres and S. M. Fuchs, FEBS letters, 2013, 587, 1247-1257.
4. G. L. Cantoni, Journal of the American Chemical Society, 1952, 74, 2942-2943.
5. J. Axelrod and R. Tomchick, The Journal of biological chemistry, 1958, 233, 702-5.
6. G. L. Cantoni, The Journal of biological chemistry, 1951, 189, 203-216.
7. J. Song, M. Teplova, S. Ishibe-Murakami and D. J. Patel, Science, 2012, 335, 709-12.
8. S. S. Wolf, Cellular and molecular life sciences: CMLS, 2009, 66, 2109-2121.
9. Y. Yang and M. T. Bedford, Nature reviews. Cancer, 2013, 13, 37-50.
10. A. J. Ruthenburg, C. D. Allis and J. Wysocka, Molecular cell, 2007, 25, 15-30.
11. H. Chen, B. Lorton, V. Gupta and D. Shechter, Oncogene, 2016, DOI: 10.1038/onc.2016.205.
12. P. Chi, C. D. Allis and G. G. Wang, Nature reviews. Cancer, 2010, 10, 457-469.
13. C. Sawan and Z. Herceg, Advances in genetics, 2010, 70, 57-85.
14. X. Bao, S. Zhao, T. Liu, Y. Liu, Y. Liu and X. Yang, The journal of histochemistry and cytochemistry: official journal of the Histochemistry Society, 2013, 61, 206-217.
15. K. Mathioudaki, A. Scorilas, A. Ardavanis, P. Lymberi, E. Tsiambas, M. Devetzi, A. Apostolaki and M. Talieri, Tumour biology: the journal of the International Society for Oncodevelopmental Biology and Medicine, 2011, 32, 575-582.
16. C. Milite, A. Feoli, M. Viviano, D. Rescigno, A. Cianciulli, A. L. Balzano, A. Mai, S. Castellano and G. Sbardella, Clinical epigenetics, 2016, 8, 102.
17. A. T. Nguyen and Y. Zhang, Genes & development, 2011, 25, 1345-1358.
18. L. Wang, Z. Zhao, M. B. Meyer, S. Saha, M. Yu, A. Guo, K. B. Wisinski, W. Huang, W. Cai, J. W. Pike, M. Yuan, P. Ahlquist and W. Xu, Cancer cell, 2014, 25, 21-36.
19. S. R. Daigle, E. J. Olhava, C. A. Therkelsen, A. Basavapathruni, L. Jin, P. A. Boriack-Sjodin, C. J. Allain, C. R. Klaus, A. Raimondi, M. P. Scott, N. J. Waters, R. Chesworth, M. P. Moyer, R. A. Copeland, V. M. Richon and R. M. Pollock, Blood, 2013, 122, 1017-1025.
20. E. Chan-Penebre, K. G. Kuplast, C. R. Majer, P. A. Boriack-Sjodin, T. J. Wigle, L. D. Johnston, N. Rioux, M. J. Munchhof, L. Jin, S. L. Jacques, K. A. West, T. Lingaraj, K. Stickland, S. A. Ribich, A. Raimondi, M. P. Scott, N. J. Waters, R. M. Pollock, J. J. Smith, O. Barbash, M. Pappalardi, T. F. Ho, K. Nurse, K. P. Oza, K. T. Gallagher, R. Kruger, M. P. Moyer, R. A. Copeland, R. Chesworth and K. W. Duncan, Nature chemical biology, 2015, 11, 432-437.
21. A. Finley and R. A. Copeland, Chemistry & biology, 2014, 21, 1196-1210.
22. H. Gowher and A. Jeltsch, Journal of molecular biology, 2001, 309, 1201-1208.
23. M. S. Kareta, Z. M. Botello, J. J. Ennis, C. Chou and F. Chedin, The Journal of biological chemistry, 2006, 281, 25893-25902.
24. B. B. Suh-Lailam and J. M. Hevel, Analytical biochemistry, 2010, 398, 218-224.
25. C. Wilczek, R. Chitta, E. Woo, J. Shabanowitz, B. T. Chait, D. F. Hunt and D. Shechter, The Journal of biological chemistry, 2011, 286, 42221-42231.
26. N. Gauthier, M. Caron, L. Pedro, M. Arcand, J. Blouin, A. Labonte, C. Normand, V. Paquet, A. Rodenbrock, M. Roy, N. Rouleau, L. Beaudet, J. Padros and R. Rodriguez-Suarez, Journal of biomolecular screening, 2012, 17, 49-58.
27. K. Devkota, B. Lohse, C. N. Jakobsen, J. Berthelsen and R. P. Clausen, Analytical biochemistry, 2015, 476, 78-80.
28. G. Ibanez, J. L. McBean, Y. M. Astudillo and M. Luo, Analytical biochemistry, 2010, 401, 203-210.
29. I. Hemeon, J. A. Gutierrez, M. C. Ho and V. L. Schramm, Analytical chemistry, 2011, 83, 4996-5004.
30. K. M. Dorgan, W. L. Wooderchak, D. P. Wynn, E. L. Karschner, J. F. Alfaro, Y. Cui, Z. S. Zhou and J. M. Hevel, Analytical biochemistry, 2006, 350, 249-255.
31. S. Duchin, Z. Vershinin, D. Levy and A. Aharoni, Epigenetics & chromatin, 2015, 8, 56.
32. C. L. Hendricks, J. R. Ross, E. Pichersky, J. P. Noel and Z. S. Zhou, Analytical biochemistry, 2004, 326, 100-105.
33. E. Collazo, J. F. Couture, S. Bulfer and R. C. Trievel, Analytical biochemistry, 2005, 342, 86-92.
34. C. Wang, S. Leffler, D. H. Thompson and C. A. Hrycyna, Biochemical and biophysical research communications, 2005, 331, 351-356.
35. T. A. Klink, M. Staeben, K. Twesten, A. L. Kopp, M. Kumar, R. S. Dunn, C. A. Pinchard, K. M. Kleman-Leyer, M. Klumpp and R. G. Lowery, Journal of biomolecular screening, 2012, 17, 59-70.
36. K. M. Drake, V. G. Watson, A. Kisielewski, R. Glynn and A. D. Napper, Assay and drug development technologies, 2014, 12, 258-271.
37. E. S. Burgos, S. A. Gulab, M. B. Cassera and V. L. Schramm, Analytical chemistry, 2012, 84, 3593-3598.
38. T. L. Graves, Y. Zhang and J. E. Scott, Analytical biochemistry, 2008, 373, 296-306.
39. J. K. Coward and F. Y. Wu, Analytical biochemistry, 1973, 55, 406-410.
40. F. Schlenk, C. R. Zydek-Cwick and N. K. Hutson, Archives of biochemistry and biophysics, 1971, 142, 144-149.
41. J. C. Hermann, R. Marti-Arbona, A. A. Fedorov, E. Fedorov, S. C. Almo, B. K. Shoichet and F. M. Raushel, Nature, 2007, 448, 775-779.
42. R. Guan, M. C. Ho, R. F. Frohlich, P. C. Tyler, S. C. Almo and V. L. Schramm, Biochemistry, 2012, 51, 9094-9103.
43. D. S. Hitchcock, H. Fan, J. Kim, M. Vetting, B. Hillerich, R. D. Seidel, S. C. Almo, B. K. Shoichet, A. Sali and F. M. Raushel, Journal of the American Chemical Society, 2013, 135, 13927-13933.
44. R. T. Borchardt, J. A. Huber and Y. S. Wu, J Med Chem, 1974, 17, 868-873.
45. K. Thomas, A. M. Haapalainen, E. S. Burgos, G. B. Evans, P. C. Tyler, S. Gulab, R. Guan and V. L. Schramm, Biochemistry, 2012, 51, 7541-7550.
46. E. S. Burgos, C. Wilczek, T. Onikubo, J. B. Bonanno, J. Jansong, U. Reimer and D. Shechter, The Journal of biological chemistry, 2015, 290, 9674-9689.
47. J. J. Christensen, J. H. Rytting and R. M. Izatt, Biochemistry, 1970, 9, 4907-4913.
48. J. Saevels, A. Zanoletty Perez, A. Salvat Jauma, A. Van Schepdael and J. Hoogmartens, Chromatographia, 1998, 47, 225-229.
49. G. Cercignani, Analytical biochemistry, 1987, 166, 418-423.
50. J. Wierzchowski, B. Wielgus-Kutrowska and D. Shugar, Biochimica et biophysica acta, 1996, 1290, 9-17.
51. J. Wierzchowski, J. M. Antosiewicz and D. Shugar, Molecular bioSystems, 2014, 10, 2756-2774.
52. J. F. Morrison and C. T. Walsh, Advances in enzymology and related areas of molecular biology, 1988, 61, 201-301.
53. H. Gowher, X. Zhang, X. Cheng and A. Jeltsch, Analytical biochemistry, 2005, 342, 287-291.
54. J. G. McCoy, L. J. Bailey, Y. H. Ng, C. A. Bingman, R. Wrobel, A. P. Weber, B. G. Fox and G. N. Phillips, Jr., Proteins, 2009, 74, 368-377.
55. Y. Wang, H. Akiyama, K. Terakado and T. Nakatsu, Scientific reports, 2013, 3, 2490.
56. V. Vanek, M. Budesinsky, P. Kabeleova, M. Sanda, M. Kozisek, I. Hanclova, J. Mladkova, J. Brynda, I. Rosenberg, M. Koutmos, T. A. Garrow and J. Jiracek, J Med Chem, 2009, 52, 3652-3665.
57. A. Sakamoto and N. Murata, Plant, cell & environment, 2002, 25, 163-171.
58. A. Nyyssola, T. Reinikainen and M. Leisola, Applied and environmental microbiology, 2001, 67, 2044-2050.
59. R. Waditee, Y. Tanaka, K. Aoki, T. Hibino, H. Jikuya, J. Takano, T. Takabe and T. Takabe, The Journal of biological chemistry, 2003, 278, 4932-4942.
60. J. P. Kallio, J. Janis, A. Nyyssola, N. Hakulinen and J. Rouvinen, Acta crystallographica. Section F, Structural biology and crystallization communications, 2009, 65, 805-808.
61. Y. R. Lee, T. S. Lin, S. J. Lai, M. S. Liu, M. C. Lai and N. L. Chan, Scientific reports, 2016, 6, 38071.
62. T. F. Smith, C. Gaitatzes, K. Saxena and E. J. Neer, Trends in biochemical sciences, 1999, 24, 181-185.
63. E. W. Debler, K. Jain, R. A. Warmack, Y. Feng, S. G. Clarke, G. Blobel and P. Stavropoulos, Proceedings of the National Academy of Sciences of the United States of America, 2016, 113, 2068-2073.
64. J. H. Zhang, T. D. Chung and K. R. Oldenburg, Journal of biomolecular screening, 1999, 4, 67-73.
65. K. Fabianowska-Majewska, J. A. Duley and H. A. Simmonds, Biochemical pharmacology, 1994, 48, 897-903.
66. O. M. Ottink, F. H. Nelissen, Y. Derks, S. S. Wijmenga and H. A. Heus, Analytical biochemistry, 2010, 396, 280-283.
67. S. S. Kamat, E. S. Burgos and F. M. Raushel, Biochemistry, 2013, 52, 7366-7368.
68. R. M. McCarty, C. Krebs and V. Bandarian, Biochemistry, 2013, 52, 188-198.
69. B. R. Branchini, M. H. Murtiashaw, R. A. Magyar and S. M. Anderson, Biochemistry, 2000, 39, 5433-5440.
70. M. B. Cassera, M. C. Ho, E. F. Merino, E. S. Burgos, A. Rinaldo-Matthis, S. C. Almo and V. L. Schramm, Biochemistry, 2011, 50, 1885-1893.
71. Z. J. Lu and G. D. Markham, The Journal of biological chemistry, 2002, 277, 16624-16631.
72. F. Garrido, C. Alfonso, J. C. Taylor, G. D. Markham and M. A. Pajares, Biochimica et biophysica acta, 2009, 1794, 1082-1090.
73. J. G. McCoy, L. J. Bailey, Y. H. Ng, C. A. Bingman, R. Wrobel, A. P. Weber, B. G. Fox and G. N. Phillips, Jr., Proteins, 2009, 74, 368-377.
74. L. Sun, M. Wang, Z. Lv, N. Yang, Y. Liu, S. Bao, W. Gong and R. M. Xu, Proceedings of the National Academy of Sciences of the United States of America, 2011, 108, 20538-20543.
75. M. Wang, R. M. Xu and P. R. Thompson, Biochemistry, 2013, 52, 5430-5440.
76. E. W. Debler, K. Jain, R. A. Warmack, Y. Feng, S. G. Clarke, G. Blobel and P. Stavropoulos, Proceedings of the National Academy of Sciences of the United States of America, 2016, 113, 2068-2073.
77. X. Zhang, H. Tamaru, S. I. Khan, J. R. Horton, L. J. Keefe, E. U. Selker and X. Cheng, Cell, 2002, 111, 117-127.
78. E. S. Burgos, C. Wilczek, T. Onikubo, J. B. Bonanno, J. Jansong, U. Reimer and D. Shechter, The Journal of biological chemistry, 2015, 290, 9674-9689.
79. J. C. Hermann, R. Marti-Arbona, A. A. Fedorov, E. Fedorov, S. C. Almo, B. K. Shoichet and F. M. Raushel, Nature, 2007, 448, 775-779.
80. A. M. Haapalainen, K. Thomas, P. C. Tyler, G. B. Evans, S. C. Almo and V. L. Schramm, Structure, 2013, 21, 963-974.
81. Y. Wang, H. Akiyama, K. Terakado and T. Nakatsu, Scientific reports, 2013, 3, 2490.
82. G. Cercignani, Analytical biochemistry, 1987, 166, 418-423.
83. J. H. Zhang, T. D. Chung and K. R. Oldenburg, Journal of biomolecular screening, 1999, 4, 67-73.
84. J. F. Morrison, Biochimica et biophysica acta, 1969, 185, 269-286.
85. I. H. Segel, Enzyme kinetics. Behavior and analysis of rapid equilibrium and steady-state enzyme systems, John Wiley & Sons, Inc., New York, 1993.

1. A method of measuring activity of a methyltransferase, comprising contacting the methyltransferase with S-adenosyl-L-methionine (SAM) and/or a fluorescent SAM analog to generate S-adenosyl-L-homocysteine (SAH), quantitatively catabolizing the SAH to S-inosyl-L-homocysteine (SIH) in the presence of deaminase TM0936, and measuring the SIH produced.

2. The method of claim 1, wherein the methyltransferase is a protein methyltransferase, a DNA methyltransferase or an RNA methyltransferase.

3. The method of claim 1, wherein the methyltransferase is a lysine methyltransferase, an arginine methyltransferase, a histone-lysine N-methyltransferase, a glycine N-methyltransferase or a sarcosine/dimethylglycine N-methyltransferase.

4. The method of claim 1, wherein the method is carried out at a pH between pH 5 and pH 10.

5. The method of claim 1, wherein the method is carried out in the presence of sinefungin.

6. The method of claim 1, wherein the methyltransferase activity is quantified by measuring a decrease of absorbance at 263 nm.

7. The method of claim 1, wherein the SAM is a fluorescent SAM analog.

8. The method of claim 7, wherein the fluorescent SAM analog is S-8-aza-adenosine-L-methionine (8-aza-SAM), and wherein the methyltransferase activity is quantified by measuring a decrease of fluorescence emission at 360 nm.

9. The method of claim 1, wherein the methyltransferase activity is measured from or within a biological sample.

10. A method of screening for an inhibitor of a methyltransferase, comprising carrying out the method of claim 1 in the presence and in the absence of a candidate compound, wherein a decrease in the activity of methyltransferase in the presence of the candidate compound, compared to methyltransferase activity in the absence of the candidate compound, indicates that the candidate compound is an inhibitor of the methyltransferase.

11. A kit for measuring the activity of a methyltransferase or a radical SAM enzyme, the kit comprising:
S-adenosyl-L-methionine (SAM) and/or fluorescent SAM analog; and
deaminase TM0936.

12. The kit of claim 11, wherein the fluorescent SAM analog is S-8-aza-adenosine-L-methionine (8-aza-SAM).

13. A method of measuring activity of a radical SAM enzyme, comprising contacting the radical SAM enzyme with S-adenosyl-L-methionine (SAM) to generate 5′-deoxyadenosine (5DOA), quantitatively catabolizing 5DOA to 5′-deoxyinosine (5DOI) in the presence of the deaminase PA3170, and measuring the 5DOI produced.

14. The method of claim 13, wherein the method is carried out at a pH between pH 5 and pH 10.

15. The method of claim 13, wherein the reaction is carried out in the presence of sinefungin.

16. The method of claim 13, wherein the radical SAM enzyme activity is quantified by measuring a decrease of absorbance at 263 nm.

17. The method of claim 13, wherein the radical SAM enzyme activity is measured from or within a biological sample.

18. A method of screening for an inhibitor of a radical SAM enzyme, comprising carrying out the method of claim 13 in the presence and in the absence of a candidate compound, wherein a decrease in the activity of radical SAM enzyme in the presence of the candidate compound, compared to radical SAM enzyme activity in the absence of the candidate compound, indicates that the candidate compound is an inhibitor of the radical SAM enzyme.

19. A kit for measuring the activity of a radical SAM enzyme, the kit comprising:
S-adenosyl-L-methionine (SAM) and
deaminase PA3170.

20. The method of claim 1, wherein the contacting and catabolizing are done as a one-step method.

21. The method of claim 20, wherein the method is a high throughput screening method.


SEQUENCE LISTING (2 sequences)

SEQ ID NO: 1 - protein, 406 aa, Thermotoga maritima
MIIGNCLILKDFSSEPFWGAVEIENGTIKRVLQGEVKVDLDLSGKLVMPALFNTHTHAPMTLLRGVAEDLSFEEWLFSKVLPIEDRLTEKMAYYGTILAQMEMARHGIAGFVDMYFHEEWIAKAVRDFGMRALLTRGLVDSNGDDGGRLEENLKLYNEWNGFEGRIFVGFGPHSPYLCSEEYLKRVFDTAKSLNAPVTIHLYETSKEEYDLEDILNIGLKEVKTIAAHCVHLPERYFGVLKDIPFFVSHNPASNLKLGNGIAPVQRMIEHGMKVTLGTDGAASNNSLNLFFEMRLASLLQKAQNPRNLDVNTCLKMVTYDGAQAMGFKSGKIEEGWNADLVVIDLDLPEMFPVQNIKNHLVHAFSGEVFATMVAGKWIYFDGEYPTIDSEEVKRELARIEKELYSS

SEQ ID NO: 2 - protein, 444 aa, Pseudomonas aeruginosa
MPNVRNPFDLLLLPTWIVPVEPAGVVLRDHALGIRDGQIAVVAPREQAMRHGATEIRELPGMLLAPGLVNAHGHSAMSLFRGLADDLPLMTWLQDHIWPAEGQWVSEDFIRDGTELAIAEQVKGGITCFSDMYFYPQAICGVVHDSGVRAQVAIPVLDFPIPGARDSAEAIRQGMALFDDLKHHPRIRIAFGPHAPYTVSDDKLEQILVLTEELDASIQMHVHETAFEVEQATERNGERPLARLHRLGLLGPRFQAVHMTQVDDDDLAMLVETNSSVIHCPESNLKLASGFCPVEKLWQAGVNVAIGTDGAASNNDLDLLGETRTAALLAKAVYGQATALDAHRALRMATLNGARALGLERLIGSLEAGKAADLVAFDLSGLAQQPVYDPVSQLIYASGRDCVRHVWVGGRQLLDDGRLLRHDEQRLIARAREWGPKIAASDRS